SEQUENCE LISTING (18 sequences)

SEQ ID NO: 1 - DNA, 20 bp, Artificial Sequence
  Synthetic primer
gtggccttctggagactgag

SEQ ID NO: 2 - DNA, 20 bp, Artificial Sequence
  Synthetic primer
catgatggcacgaacctgta

SEQ ID NO: 3 - DNA, 20 bp, Artificial Sequence
  Synthetic primer
aacatccccattctaaggca

SEQ ID NO: 4 - DNA, 20 bp, Artificial Sequence
  Synthetic primer
gccttgctttcccctttaat

SEQ ID NO: 5 - DNA, 23 bp, Artificial Sequence
  Synthetic primer
ctgggtcactggtaactgacatt

SEQ ID NO: 6 - DNA, 21 bp, Artificial Sequence
  Synthetic primer
gtcctacctttacaggggatg

SEQ ID NO: 7 - DNA, 20 bp, Artificial Sequence
  Synthetic primer
tttcaaacaccatgacccaa

SEQ ID NO: 8 - DNA, 20 bp, Artificial Sequence
  Synthetic primer
tgctgacctgctggattaca

SEQ ID NO: 9 - DNA, 20 bp, Artificial Sequence
  Synthetic primer
ctgcattgttttgccagtgt

SEQ ID NO: 10 - DNA, 20 bp, Artificial Sequence
  Synthetic primer
actgctcactcacccacctc

SEQ ID NO: 11 - DNA, 20 bp, Artificial Sequence
  Synthetic primer
ctgcaagttgggaagaagga

SEQ ID NO: 12 - DNA, 20 bp, Artificial Sequence
  Synthetic primer
tgtgcctctttccaactcct

SEQ ID NO: 13 - DNA, 20 bp, Artificial Sequence
  Synthetic primer
tgtggtggaccagaccaata

SEQ ID NO: 14 - DNA, 20 bp, Artificial Sequence
  Synthetic primer
tgactccatcattcccacag

SEQ ID NO: 15 - DNA, 21 bp, Artificial Sequence
  Synthetic primer
ccagactgttccaacatcacc

SEQ ID NO: 16 - DNA, 20 bp, Artificial Sequence
  Synthetic primer
ctagttccggctgctctttg

SEQ ID NO: 17 - DNA, 20 bp, Artificial Sequence
  Synthetic primer
gctacagcttcaccaccaca

SEQ ID NO: 18 - DNA, 20 bp, Artificial Sequence
  Synthetic primer
aaggaaggctggaaaagagc

(19) United States
(12) Patent Application Publication
(10) Pub. No.: US 2007/0135336 A1
(43) Pub. Date: Jun. 14, 2007

(54) Follistatin isoforms and uses thereof

(76) Inventors: David De Kretser, Surrey Hills (AU); John Roderick Morrison, Caulfield North (AU); Shyr-Yeu Lin, Taipei (TW)

Correspondence Address: SCULLY, SCOTT, MURPHY & PRESSER, P.C., 400 GARDEN CITY PLAZA, SUITE 300, GARDEN CITY, NY, 11530, US

(21) Appl. No.: 10/571,837

(22) PCT Filed: Sep. 15, 2004
(86) PCT No.: PCT/AU04/01253

§ 371 Date: Nov. 13, 2006

(30) Foreign Application Priority Data
Sep. 15, 2003 (AU) 2003905010

(51) Int. Cl. A61K 38/22

(52) U.S. Cl. 514/12

(57) ABSTRACT

The present invention relates generally to regulating biological developmental process such as the biological functions involved in such developmental processes including growth and survival of an animal and particularly to methods of modifying the developmental processes and biological functions in cells. The invention also relates to methods of preventing and treating biological-development related conditions by regulating the developmental processes and modifying the biological functions in the cells.

### FIELD OF THE INVENTION

The present invention relates generally to a method of modulating biological developmental processes and functions in a mammal and to agents useful for same. More particularly the present invention relates to a method of modulating biological developmental processes in a mammal by modulating the functional activity of the follistatin isoforms. The present invention is useful, inter alia, in modulating the developmental processes and biological functions of cells, such as in the context of modulating the growth and survival of an animal. The invention also relates to methods of preventing and treating biological development related conditions by regulating developmental processes and/or modifying the biological functions of cells.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

The processes involved in biological development are complex and involve the interaction of a multitude of proteins. Any adverse impact on one or more of these proteins will modulate the signalling cascade which ensues from these interactions, thereby resulting in modulation of the development of a cell or group of cells. This can necessarily impact on one or more aspects of the normal development of a mammal, where such impacts occur during crucial developmental processes.

Of the many proteins which are involved in biological development, one particularly significant family of growth factors, the TGF-β superfamily, show diverse functionality during embryonic development and adult tissue homeostasis. These proteins can essentially be grouped into four families: the TGF-β family, the activin family, the bone morphogenetic protein (BMP) family, and the growth differentiation factor (GDF) family.

Follistatin is a glycosylated single-chain protein which is functionally linked to members of the TGF-β superfamily. It was originally isolated from ovarian follicular fluid on the basis of suppression of FSH secretion by pituitary cells. Subsequently, follistatin was shown to function as an activin-binding protein with the capacity to neutralize the majority of the actions of the activin.

While the isolation and characterization of follistatin as an activin binding protein has been known for more than ten years, many aspects of the biology of follistatin still continue to emerge from ongoing research. Certainly, many of the biological effects of follistatin can be explained by its capacity to bind and neutralize the actions of activin. Indeed, the widespread effects of activin in a diversity of systems have highlighted the significance of follistatin biology and many studies indicate that these actions of follistatin involve autocrine/paracrine processes affecting the physiology of many systems such as reproduction, development, vascular biology, inflammation, fibrosis and wound healing (Chang et al., Endocr Rev 23 787-823, 2002; Welt et al., Exp Biol Med (Maywood) 227 724-752, 2002; Lin et al., Reproduction 126 133-148, 2003)

Recent papers, however, have considerably broadened the field of follistatin biology. Emerging and compelling evidence supports the interaction of follistatin with some of the TGF-13 superfamily members other than activin. For example, BMP-2, 4, 7 and 15, myostatin (GDF-8) and GDF-11 have been implicated as targets to which follistatin can bind (Iemura et al., Proceedings of the National Academy of Sciences of the United States of America 95 9337-9342, 1998; Lee & McPherran, Proceedings of the National Academy of Sciences of the United States of America 98 9306-9311, 2001; Otsuka et al., Biochem Biophys Res Commun 289 961-966, 2001). Furthermore, the signalling pathways of the TGF-β superfamily members have been demonstrated to cross-talk with the signalling pathways of other families, complicating the potential influence of follistatin in normal biological functions.

Accordingly, in light of the complexity of the developmental processes to which all mammals are subject, there is an ongoing need to elucidate the mechanisms by which the various developmental pathways are regulated. In work leading up to the present invention it has been surprisingly determined not only that follistatin plays a crucial role in cellular and tissue development, but that the follistatin isoforms, 315 and 288, exhibit quite distinct roles in this regard. Until the advent of the present invention the precise role of follistatin in the context of many biological functioning and developmental processes was not fully understood. However, the findings of the present invention have now facilitated the development of methodology directed to modulating these processes, in a directed manner, by modulating the levels of one or both of the follistatin isoforms. Accordingly, there are now provided both methods for the therapeutic or prophylactic treatment of conditions characterised by inappropriate or deficient biological functioning or development and means for screening for regulators of the functioning of follistatin 315 and/or 288.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The subject specification contains nucleotide sequence information prepared using the programme PatentIn Version 3.1, presented herein after the bibliography. Each nucleotide sequence is identified in the sequence listing by the numeric indicator <210> followed by the sequence identifier (eg. <210>1, <210>2, etc). The length, type of sequence (DNA, etc) and source organism for each sequence is indicated by information provided in the numeric indicator fields <211>, <212> and <213>, respectively. Nucleotide sequences referred to in the specification are identified by the indicator SEQ ID NO: followed by the sequence identifier (eg. SEQ ID NO:1, SEQ ID NO:2, etc.). The sequence identifier referred to in the specification correlates to the information provided in numeric indicator field <400> in the sequence listing, which is followed by the sequence identifier (eg. <400>1, <400>2, etc). That is SEQ ID NO:1 as detailed in the specification correlates to the sequence indicated as <400>1 in the sequence listing

Accordingly, one aspect of the present invention is directed to a method of modulating the biological functioning and/or development of an animal or a cell, said method comprising modulating the functionally effective level of one or more follistatin isoforms or derivative, fragment, homologue, mutant or variant thereof.

More particularly, the present invention is directed to a method of modulating the biological functioning and/or development of a mammal or a cell, said method comprising modulating the functionally effective level of one or both of follistatin 315 and/or follistatin 288 or derivative, fragment, homologue, mutant or variant thereof. Preferably, the subject biological functioning and/or development are:

(i) cellular or animal growth and survival;

(ii) capacity for movement (in the context of an animal);

(iii) skin and hair follicle development;

(iv) fetal development;

(v) embryogenesis; or

(vi) development of genital tubercles.

Still more preferably the subject biological functioning and/or development are upregulated by upregulation in the level of follistatin 315 and, optionally, the concurrent downregulation in the level of follistatin 288.

In another preferred embodiment, the subject biological functioning and/or development are vasculogenesis or angiogenesis. Still more preferably, the subject biological functioning and/or development are upregulated by the upregulation in the level of follistatin 288 and, optionally, the concurrent downregulation in the level of follistatin 315.

In a related aspect of the present invention there is provided a cell characterised by modulated biological functioning, said cell comprising modulated expression and/or activity of follistatin isoform 315 and/or 288.

In yet another aspect of the present invention there is provided a transgenic animal characterised by modulated biological functioning and/or development, said animal comprising modulated expression and/or activity of follistatin isoform 315 and/or 288.

The present invention therefore contemplates a method for therapeutically and/or prophylactically treating a development-related condition or a predisposition to the onset of a development related condition in a mammal, said method comprising modulating the functionally effective level of one or more follistatin isoforms, or derivative, fragment, homologue, mutant or variant thereof, in said mammal.

More particularly, the present invention therefore contemplates a method of therapeutically and/or prophylactically treating a development-related condition or a predisposition to the onset of a development related condition in a mammal, said method comprising modulating the functionally effective level of one or both of follistatin 315 or follistatin 288, or derivative, fragment, homologue, mutant or variant thereof, in said mammal.

Yet another aspect of the present invention relates to the use of an agent capable of modulating the functionally effective level of one or more follistatin isoforms, or derivative, fragment, homologue, mutant or variant thereof, in the manufacture of a medicament for the therapeutic and/or prophylactic treatment of a development-related condition, or a predisposition to the onset of a development related condition in a mammal.

Still a further aspect of the present invention relates to the use of an agent capable of modulating the functionally effective level of one or both of follistatin 315 or follistatin 288 or derivative, fragment, homologue, mutant or variant thereof, in the manufacture of a medicament for the therapeutic and/or prophylactic treatment of a development-related condition, or a predisposition to the onset of a development related condition in a mammal.

In yet another further aspect, the present invention contemplates a pharmaceutical composition comprising the modulatory agent as hereinbefore defined together with one or more pharmaceutically acceptable carriers and/or diluents. Said agents are referred to as the active ingredients.

### BRIEF DESCRIPTION OF THE DRAWINGS

FIG. 1 is an image of the genotype evidence for detecting transgenic founders. The figure shows the genotyping evidence used to detect transgenic founders. (−) indicates the negative control. (+) indicates the positive control. Four pairs of PCR primers, 10 kb-UP-AF & R, 5 kb-UP-AF & R, Southern. F & R and 1.5 kb-DOWN-AF & R, are characterized in Table 5.1. (a)˜(d) are the gel pictures for screening the transgenic founders carrying the pNEB-FS vector. (e)˜(h) are the gel pictures for screening the transgenic founders carrying the PAC-FS vector. (a) & (e) used the primer pair of 10 kb-UP-AF & R; (b) & (f) used the primer pair of 5 kb-UP-AF & R; (c) & (g) used the primer pair of Southern. F & R; (d) & (h) used the primer pair of 1.5 kb-DOWN-AF & R. Thus there are three founders from the pNEB-FS vector and two founders from the PAC-FS vector.

FIG. 2 is an image of the skin of fs-ko, wt, FS²⁵-ko.1, FS²⁵-ko.2, FS⁹⁵-ko.1 and FS⁹⁵-ko.2 at birth. The pictures show the skin of fs-ko, wt and four lines of the rescued mice on day 0 postpartum, at 10× magnification. Comparison of the wt skin with the fs-ko skin revealed that the whole layer of the wt epidermis appeared to be thicker while the strata corneum and granulosum epidermis of fs-ko seemed to be decreased compared to those of wt. Moreover, all four lines of the rescued mice had skin that was more similar to that of fs-ko although sometimes the skin of FS⁹⁵-ko.1 looked more similar to that of wt.

FIG. 3 is an image of the results of genotyping for detecting transgenic founders. The figure shows the genotyping evidence for detecting transgenic founders. (−) means the negative control. (+) means the positive control. Four pairs of PCR primers, 10 kb-UP-AF & R, 5 kb-UP-AF & R, Southern. F & R and 1.5 kb-DOWN-AF & R, are targeting the sites 10 kb upstream of, 5 kb upstream of, within, and 1.5 kb downstream of the human follistatin gene, respectively. (a)˜(d) are the gel pictures for screening the transgenic founders carrying the PAC-FSm1 vector. (e)˜(h) are the gel pictures for the screening the transgenic founders carrying the PAC-FSm2 vector. (a) & (e) are using the primer pair of 10 kb-UP-AF & R; (b) & (f) are using the primer pair of 5 kb-UP-AF & R; (c) & (g) are using the primer pair of Southern. F & R; (d) & (h) are using the primer pair of 1.5 kb-DOWN-AF & R. Thus, there are three founders from the PAC-FSm1 vector and one founder from the PAC-FSm2 vector.

### DETAILED DESCRIPTION OF THE INVENTION

The present invention is predicated, in part, on the surprising determination that the two isoforms of follistatin play crucial but quite distinct roles in mammalian biological functional and/or developmental processes. Accordingly, these findings have now facilitated the rational design of means for modulating such processes and, in particular for therapeutically or prophylactically treating conditions which are characterised by an aberrant or inappropriate process, such as in the context of growth, development of hair and skin follicles, vasculogenesis and/or angiogenesis.

Accordingly, one aspect of the present invention is directed to a method of modulating the biological functioning and/or development of an animal or a cell, said method comprising modulating the functionally effective level of one or more follistatin isoforms or derivative, fragment, homologue, mutant or variant thereof.

More particularly, the present invention is directed to a method of modulating the biological functioning and/or development of a mammal or a cell, said method comprising modulating the functionally effective level of one or both of follistatin 315 and/or follistatin 288 or derivative, fragment, homologue, mutant or variant thereof.

Without limiting the present invention to any one theory or mode of action, it has been surprisingly determined that mice carrying the follistatin 315 gene, alone, can survive. This is in contrast to mice carrying only the follistatin 288 gene. Accordingly, these two follistatin isoforms exhibit distinct impacts on the development process. Mice carrying and expressing both the 288 and the 315 follistatin isoforms also failed to survive. More specifically, mice expressing follistatin 315, alone, displayed much improvement in the growth retardation which is observed in mice expressing both isoforms of follistatin, as well as increasing their capacity for movement.

The expression of follistatin 315 has also been determined to be crucial in terms of the regulation of apoptosis vs cellular proliferation in addition to the normal development of skin and hair follicles. Further, in mice expressing the follistatin 288 isoform alone, the upregulation of vasculogenesis and/or angiogenesis has been facilitated.

Reference to “biological functioning” should therefore be understood as a reference to all functions that are appropriate for and lead to the survival and development of the cell and animal and may include, but are not limited to, the balance between two opposing biological processes such as apoptosis and proliferation, differentiation, homeostasis, appendage development, cell growth and regeneration, cell signalling such as transcriptional and translational processes regulated by activin, hormone balance, maturation of cells. In the context of this application, reference to “biological functioning” also incorporates biological developmental processes. “Biological development”, in this regard, refers to the maintenance and advancement of the animal as a whole which preferably leads to growth and survival of the animal. More preferably, biological development includes improvements in growth or a capacity for movement, vascularization, angiogenesis, avoidance of growth retardation, fetal development, embryogenesis or development of genital tubercles.

The cells in which biological function may be modulated include cells in which follistatin is present and may include, but are not limited to, cells of the reproductive system, urogenital system, endocrine system, neural system, digestive system, or hematopoietic system. Preferably the cells are cells of the testis, ovary, uterus, kidney, prostate, pituitary, pancreas, forebrain, cerebellum, spinal cord, salivary glands, liver, stomach, bone, thymus, heart, blood vessels, lung, muscle, skin and eye. The cell may be a stem cell or an ES cell. More preferably they are vessel cells, blood cells, spermatocytes, oocytes, epithelial cells, skin cells, hair follicles, lung cells, hepatic cells, neural cells, islet cells, kidney, Sertoli cells, granulosa cells or germ cells.

It should also be understood that the cell which is the subject of modulation in accordance with the method of the invention may be an isolated cell or a cell which forms part of a group of cells, such as an isolated tissue. The cell may also be localised in a mammal, that is it is not isolated, therefore requiring the subject method to be performed in vivo. Where the subject cell is one of a group of cells or a tissue, either isolated or not, the subject method may modulate the functioning of all the cells in that group or just a subgroup of cells in that group. Similarly, in the context of the modulation of the biological functioning or development of a “mammal”, it should be understood that the subject modulation may be achieved in the context of modulating the levels of follistatin isoforms either systemically or in a localised manner. Still further, irrespective of which means is employed, the cellular impact of the change in follistatin isoform levels may occur in the context of either all cells or just a subgroup of cells within the relevant environment.

Preferably, the subject biological functioning and/or development are:

(i) cellular or animal growth and survival;

(ii) capacity for movement (in the context of an animal);

(iii) skin and hair follicle development;

(iv) fetal development;

(v) embryogenesis; or

(vi) development of genital tubercles.

Still more preferably the subject biological functioning and/or development are upregulated by upregulation in the level of follistatin 315 and, optionally, the concurrent downregulation in the level of follistatin 288.

In another preferred embodiment, the subject biological functioning and/or development are vasculogenesis or angiogenesis. Still more preferably, the subject biological functioning and/or development are upregulated by the upregulation in the level of follistatin 288 and, optionally, the concurrent downregulation in the level of follistatin 315.

Still without limiting the present invention in any way, follistatin is widely expressed as two separate isoforms: follistatin-288 and follistatin-315. The biology of each isoform is poorly understood although follistatin-315 is thought to be the circulating form while follistatin-288 appears to be bound to heparin-sulphate proteoglycans. Follistatin-315 is generally expressed at approximately 20 times the level of follistatin-288. Molecular analysis of the various isoforms shows that follistatin is encoded by a single gene and the variety of molecular weights (31-39 kDa) arise from alternative splicing, glycosylation and proteolytic cleavage. Alternative splicing occurs at the 3′-terminal of the gene between exon 5 and exon 6. The splicing out of intron 5, generating a stop codon immediately following the last amino acid of exon 5, leads to the termination of the coding sequence for a precursor of 317 amino acids (pre-follistatin 317), the COOH-terminal truncated form. On the other hand, exon 6a is spliced out together with intron 5 to generate a precursor of 344 amino acids (pre-follistatin 344). Cleavage of the signal peptide (29 amino acids) generates the mature follistatin isoforms of 288 and 315 amino acids (follistatin-288 and follistatin-315).

Accordingly, reference to “follistatin 315” and “follistatin 288” should be read as including reference to all forms of follistatin 315 and follistatin 288 including, by way of example, the three protein cores and six molecular weight forms which have been identified as arising from the alternatively spliced mRNAs FS315 and FS288. It should, still further, be understood to extend to any FS315 or FS288 protein, whether existing as a monomer, multimer or fusion protein. This definition should also be understood to extend to precursor forms of these isoforms.

The determination by the inventors that the modulation of the follistatin isoforms alone or in combination with each other is central to the regulation of the biological functioning and development of cells and animals with the potential to affect development of pathological events.

Reference to “modulating” should be understood as a reference to upregulating or downregulating the biological functioning and/or development of a mammalian cell or mammal. Reference to “downregulation” in this context should be understood as a reference to preventing, reducing (e.g. slowing) or otherwise inhibiting one or more aspects of said functioning or development while reference to “upregulating” in this context should be understood to have the converse meaning.

It should be understood that in terms of modulating the biological functioning and/or development of a mammal or mammalian cell or tissue, this may be achieved either by modulating the actual levels of the follistatin isoforms or by modulating their functionality. For example, and without limiting the present invention to any one theory or mode of action, it is known that activin A binds follistatin (this molecule functioning as a follistatin antagonist). Accordingly, binding of activin A to follistatin, thereby blocking the functionality of follistatin will downregulate its activity without necessarily impacting on the actual levels of this molecule.

Reference herein to attaining either a “functionally effective level” or “functionally ineffective level” of follistatin isoform should be understood as a reference to attaining that level of follistatin isoform at which modulation of the biological function or development process can be achieved, whether that be up-regulation or down-regulation. In this regard, it is within the skill of the person of skill in the art to determine, utilising routine procedures, the threshold level of one or both follistatin isoform levels above or below which the subject process is modulated. In this regard, modulating the level of the subject follistatin isoforms includes modifying or altering the expression and/or activity of the follistatin isoform 315 and/or 288 compared to unmodified, pre-existing or natural levels, (herein referred to as “background” levels) of follistatin isoform 315 and/or 288 that exist in the subject cell or mammal. That is, the level may be modulated relative to the level of an untreated cell or animal or it may be modulated relative to the level resulting from a previous modulation event. This may occur, for example, in the context of a stepwise treatment program where consecutive treatment events are performed to progressively alter or, alternatively, ensure maintenance of a particular level of follistatin. Expression and/or activity may be increased or decreased compared to background levels.

It should be understood that reference to an “effective level” means the level necessary to at least partly attain the desired response. The amount may vary depending on the health and physical condition of the cellular population and/or individual being treated, the taxonomic group of the cellular population and/or individual being treated, the degree of up or down-regulation which is desired, the formulation of the composition which is utilised, the assessment of the medical situation and other relevant factors. Accordingly, it is expected that this level may vary between individual situations, thereby falling in a broad range, which can be determined through routine trials.

Modulating follistatin isoform 315 and/or follistatin isoform 288 (herein collectively referred to as “follistatin isoforms”) levels may be achieved by any suitable means including, but not limited to:
(i) Modulating absolute levels of the follistatin isoforms such that either more or less of the follistatin isoforms is present in the cellular environment.
(ii) Agonising or antagonising follistatin isoform protein functional activity such that the functional effectiveness of said follistatin isoform is either increased or decreased. For example, increasing the half life of a follistatin isoform may achieve an increase in the functionally effective level of the follistatin isoform without actually necessitating an increase in the absolute concentration of the follistatin isoform. Similarly, the partial antagonism of the follistatin isoform may act to reduce, although not necessarily eliminate, the functional effectiveness of said follistatin isoform.
   Accordingly, this may provide a means of down-regulating follistatin isoform functioning without necessarily down-regulating absolute concentrations of the follistatin isoforms.

In terms of achieving the up or down-regulation of the follistatin isoforms, means for achieving this objective would be well known to the person of skill in the art and include, but are not limited to:
(i) Introducing into a cell a nucleic acid molecule encoding the follistatin isoform in order to up-regulate the capacity of said cell to express the follistatin isoform.
(ii) Introducing into a cell a proteinaceous or non-proteinaceous molecule which modulates transcriptional and/or translational regulation of a gene, wherein this gene may be the follistatin isoform gene or functional portion thereof or some other gene or gene region (eg. promoter region) which directly or indirectly modulates the expression of the follistatin isoform gene.
(iii) Introducing into a cell the follistatin isoform expression product (this should be understood to include the use of follistatin isoform homologues).
(iv) Introducing a proteinaceous or non-proteinaceous molecule which functions as an antagonist to the follistatin isoform expression product (e.g. activin).
(v) Introducing a proteinaceous or non-proteinaceous molecule which functions as an agonist of the follistatin isoform expression product.

The proteinaceous molecules described above may be derived from any suitable source such as natural, recombinant or synthetic sources and includes fusion proteins or molecules which have been identified following, for example, natural product screening. The reference to non-proteinaceous molecules may be, for example, a reference to a nucleic acid molecule or it may be a molecule derived from natural sources, such as for example natural product screening, or may be a chemically synthesised molecule. The present invention contemplates analogues of the follistatin isoform expression product or small molecules capable of acting as agonists or antagonists. Chemical agonists may not necessarily be derived from the follistatin isoform expression product but may share certain conformational similarities. Alternatively, chemical agonists may be specifically designed to meet certain physiochemical properties. Antagonists may be any compound capable of blocking, inhibiting or otherwise preventing the follistatin isoform from carrying out its normal biological function. Antagonists include monoclonal antibodies and antisense nucleic acids which prevent transcription or translation of follistatin isoform genes or mRNA in mammalian cells. Modulation of expression may also be achieved utilising antigens, RNA, ribosomes, DNAzymes, aptamers, antibodies or molecules suitable for use in cosuppression. Suitable antisense oligonucleotide sequences (single stranded DNA fragments) of follistatin isoforms may be created or identified by their ability to suppress the expression of the follistatin isoforms. The production of antisense oligonucleotides for a given protein is described in, for example, Stein and Cohen, 1988 (Cancer Res 48:2659-68) and van der Krol et al., 1988 (Biotechniques 6:958-976).

The proteinaceous and non-proteinaceous molecules referred to in points (i)-(v), above, are herein collectively referred to as “modulatory agents”.

Screening for the modulatory agents hereinbefore defined can be achieved by any one of several suitable methods including, but in no way limited to, contacting a cell comprising the follistatin isoform gene or functional equivalent or derivative thereof with an agent and screening for the modulation of follistatin isoform protein production or functional activity, modulation of the expression of a nucleic acid molecule encoding the follistatin isoform or modulation of the activity or expression of a downstream follistatin isoform cellular target. Detecting such modulation can be achieved utilising techniques such as Western blotting, electrophoretic mobility shift assays and/or the readout of reporters of vi activity such as luciferases, CAT and the like.

It should be understood that the follistatin isoform genes or functional equivalents or derivatives thereof may be naturally occurring in the cell which is the subject of testing or may have been transfected into a host cell for the purpose of testing. Further, the naturally occurring or transfected gene may be constitutively expressed—thereby providing a model useful for, inter alia, screening for agents which down regulate follistatin isoform activity, at either the nucleic acid or expression product levels, or the gene may require activation—thereby providing a model useful for, inter alia, screening for agents which up-regulate follistatin isoform expression. Further, to the extent that a follistatin isoform nucleic acid molecule is transfected into a cell, that molecule may comprise the entire follistatin isoform gene or it may merely comprise a portion of the gene such as the portion which regulates expression of the follistatin isoform product. For example, the follistatin isoform promoter region may be transfected into the cell which is the subject of testing. In this regard, where only the promoter is utilised, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively. In another example, the subject of detection could be a downstream follistatin isoform regulatory target, rather than the follistatin isoform itself. Yet another example includes follistatin isoform binding sites ligated to a minimal reporter. Modulation of follistatin isoform activity can be detected by screening for the modulation of one or more aspects of cellular development. This is an example of an indirect system where modulation of follistatin isoform expression, per se, is not the subject of detection. Rather, modulation of the down-stream activity which follistatin isoform regulates is monitored.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the follistatin isoform nucleic acid molecule or expression product itself or which modulate the expression of an upstream molecule, which upstream molecule subsequently modulates follistatin isoform expression or expression product activity. Accordingly, these methods provide a mechanism of detecting agents which either directly or indirectly modulate follistatin isoform expression and/or activity.

The agents which are utilised in accordance with the method of the present invention may take any suitable form. For example, proteinaceous agents may be glycosylated or unglycosylated, phosphorylated or dephosphorylated to various degrees and/or may contain a range of other molecules fused, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound otherwise associated with any other proteinaceous or non-proteinaceous molecules. For example, in one embodiment of the present invention said agent is associated with a molecule which permits its targeting to a localised region.

The subject proteinaceous or non-proteinaceous molecule may act either directly or indirectly to modulate the expression of the follistatin isoform or the activity of the follistatin isoform expression product. Said molecule acts directly if it associates with the follistatin isoform nucleic acid molecule or expression product to modulate expression or activity, respectively. Said molecule acts indirectly if it associates with a molecule other than the follistatin isoform nucleic acid molecule or expression product which other molecule either directly or indirectly modulates the expression or activity of the follistatin isoform nucleic acid molecule or expression product, respectively. Accordingly, the method of the present invention encompasses the regulation of activin A nucleic acid molecule expression or expression product activity via the induction of a cascade of regulatory steps.

The term “expression” refers to the transcription and translation of a nucleic acid molecule. Reference to “expression product” is a reference to the product produced from the transcription and translation of a nucleic acid molecule. Reference to “modulation” should be understood as a reference to up-regulation or down-regulation.

“Derivatives” of the molecules herein described (for example the follistatin isoforms or the modulatory agents) include fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, in order to increase or otherwise enhance the rate and volume of production by that particular cellular source. Parts or fragments include, for example, active regions of the molecule. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

Derivatives also include fragments having particular regions, such as active regions, or parts of the entire protein fused to peptides, polypeptides or other proteinaceous or non-proteinaceous molecules. For example, follistatin, or derivative thereof may be fused to a molecule to facilitate its localisation to a particular site. Analogues of the molecules contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Derivatives of nucleic acid sequences which may be utilised in accordance with the method of the present invention may similarly be derived from single or multiple nucleotide substitutions, deletions and/or additions including fusion with other nucleic acid molecules. The derivatives of the nucleic acid molecules utilised in the present invention include oligonucleotides, PCR primers, antisense molecules, molecules suitable for use in cosuppression and fusion of nucleic acid molecules. Derivatives of nucleic acid sequences also include degenerate variants.

A “variant” or “mutant” of the follistatin isoform should be understood to mean molecules which exhibit at least some of the functional activity of the form of follistatin of which it is a variant or mutant. A variation or mutation may take any form and may be naturally or non-naturally occurring.

A “homologue” is meant that the molecule is derived from a species other than that which is being treated in accordance with the method of the present invention. This may occur, for example, where it is determined that a species other than that which is being treated produces a form of follistatin isoform which exhibits similar and suitable functional characteristics to that of the follistatin isoform which is naturally produced by the subject undergoing treatment.

Chemical and functional equivalents should be understood as molecules exhibiting any one or more of the functional activities of the subject molecule, which functional equivalents may be derived from any source such as being chemically synthesised or identified via screening processes such as natural product screening. For example chemical or functional equivalents can be designed and/or identified utilising well known methods such as combinatorial chemistry or high throughput screening of recombinant libraries or following natural product screening. Antagonistic agents can also be screened for utilising such methods.

For example, libraries containing small organic molecules may be screened, wherein organic molecules having a large number of specific parent group substitutions are used. A general synthetic scheme may follow published methods (eg., Bunin B A, et al. (1994) Proc. Natl. Acad. Sci. USA, 91:4708-4712; DeWitt S H, et al. (1993) Proc. Natl. Acad. Sci. USA, 90:6909-6913). Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

There is currently widespread interest in using combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. In the present context, for example, they may be used as a starting point for developing follistatin isoform analogues which exhibit properties such as more potent pharmacological effects. A follistatin isoform or a functional part thereof may according to the present invention be used in combination libraries formed by various solid-phase or solution-phase synthetic methods (see for example U.S. Pat. No. 5,763,263 and references cited therein). By use of techniques, such as that disclosed in U.S. Pat. No. 5,753,187, millions of new chemical and/or biological compounds may be routinely screened in less than a few weeks. Of the large number of compounds identified, only those exhibiting appropriate biological activity are further analysed.

With respect to high throughput library screening methods, oligomeric or small-molecule library compounds capable of interacting specifically with a selected biological agent, such as a biomolecule, a macromolecule complex, or cell, are screened utilising a combinational library device which is easily chosen by the person of skill in the art from the range of well-known methods, such as those described above. In such a method, each member of the library is screened for its ability to interact specifically with the selected agent. In practising the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

In addition to screening for molecules which mimic the activity of a follistatin isoform one may identify and utilise molecules which function agonistically or antagonistically to the follistatin isoform in order to up or down-regulate the functional activity of the follistatin isoform in relation to modulating cellular growth. The use of such molecules is described in more detail below. To the extent that the subject molecule is proteinaceous, it may be derived, for example, from natural or recombinant sources including fusion proteins or following, for example, the screening methods described above. The non-proteinaceous molecule may be, for example, a chemical or synthetic molecule which has also been identified or generated in accordance with the methodology identified above. Accordingly, the present invention contemplates the use of chemical analogues of a follistatin isoform capable of acting as agonists or antagonists. Chemical agonists may not necessarily be derived from a follistatin isoform but may share certain conformational similarities. Alternatively, chemical agonists may be specifically designed to mimic certain physiochemical properties of a follistatin isoform. Antagonists may be any compound capable of blocking, inhibiting or otherwise preventing a follistatin isoform from carrying out its normal biological functions. Antagonists include monoclonal antibodies specific for a follistatin isoform or parts of a follistatin isoform.

Analogues of a follistatin isoform or of follistatin isoform agonistic or antagonistic agents contemplated herein include, but are not limited to, modifications to side chains, incorporating unnatural amino acids and/or derivatives during peptide, polypeptide or protein synthesis and the use of crosslinkers and other methods which impose conformational constraints on the analogues. The specific form which such modifications can take will depend on whether the subject molecule is proteinaceous or non-proteinaceous. The nature and/or suitability of a particular modification can be routinely determined by the person of skill in the art.

For example, examples of side chain modifications contemplated by the present invention include modifications of amino groups such as by reductive alkylation by reaction with an aldehyde followed by reduction with NaBH4; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl--aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Crosslinkers can be used, for example, to stablise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

As detailed earlier, modulation of the follistatin isoform functional levels may be achieved via the administration of the follistatin isoform, a nucleic acid molecule encoding the follistatin isoform or an agent which effects modulation of the follistatin isoform activity or follistatin isoform gene expression (herein collectively referred to as “modulatory agents”).

In one preferred embodiment, modulation of the expression of the follistatin isoforms is achieved by directly affecting expression of the isoform in the cell. Preferably, the introduction of a construct with the gene comprising follistatin isoform 315 and/or 288 will allow for modulation of the levels of follistatin isoform 315 and/or 288 upon expression and thereby affect the biological functions for which it is directed.

Without limiting the present invention to any one theory or mode of action, any cell can accept a gene or gene construct encoding a follistatin isoform. However, ideally, the cell can readily accept a gene construct and fully integrate it into the cell to have an influence on the biological function or its own function as well as adjoining cells and cellular environment. The cell may be a stem cell, oocyte or germ cell thereby allowing for modulation of the isoforms in cells differentiated from the stem cells.

The gene for said follistatin isoform may be obtained by PCR amplification of mRNA from human (or other species) tissues using follistatin isoform specific primers and inserted into a mammalian expression vector such as pcDNA3.1 (Clontech) to form a construct or vector that may be transfected into the cell to express the follistatin isoform. Preferably, a gene sequence for the follistatin isoform is operably linked to a regulatory sequence which is capable of providing for the expression of the coding sequence by a cell. The term “operably linked” refers to a juxtaposition wherein the components described are in a relationship permitting them to function in their intended manner. A regulatory sequence “operably linked” to a coding sequence is ligated in such a way that expression of the coding sequence is achieved under conditions compatible with the control sequences. While operably linked nucleic acid sequences can be contiguous and in the same reading frame, certain genetic elements e.g. repressor genes may not be contiguously linked to the coding sequence but may still control transcription/translation of the coding sequence.

The term “regulatory sequence(s)” includes promoters and enhancers and other expression regulation signals. These may be selected to be compatible with the cell for which the expression vector is designed. Mammalian promoters, such as β-actin promoters and the myosin light chain promoter may be used. However, other promoters may be adopted to achieve the same effect. These alternate promoters are generally familiar to the skilled addressee. Mammalian promoters also include the metallothionein promoter which can upregulate expression in response to heavy metals such as cadmium and is thus an inducible promoter. Tissue-specific promoters may be used. Viral promoters may also be used, for example the Moloney murine leukaemia virus long terminal repeat (MML V L TR), the promoter rous sarcoma virus (RSV) L TR promoter, the SV40 promoter, the human cytomegalovirus (CMV) IE promoter, herpes simplex virus promoters or adenovirus promoters. All these promoters are readily available in the art.

Such vectors may be transfected into a suitable cell in which the biological function is desired to provide for expression of a polypeptide encoding a follistatin isoform which then can influence the biological function depending on the influence of isoforms 315 and/or 288 on themselves and/or surrounding tissue.

The vectors may be, for example, plasmid, virus or phage vectors provided with an origin of replication, optionally a promoter for the expression of the follistatin isoform and optionally a regulator of the promoter. The vectors may contain one or more selectable marker genes for example an ampicillin resistance gene in the case of a bacterial plasmid or a neomycin resistance gene for a mammalian vector. Vectors may be used in vitro, for example for the production of RNA or used to transfect or transform a host cell. The vector may also be adapted to be used in vivo for example in a method of gene therapy or a DNA vaccine.

The cells in which the vector is transfected is expected to provide for such post-translational modifications (eg myristolation, glycosylation, truncation, lapidation and tyrosine, serine or threonine phosphorylation) as may be needed to confer optimal biological activity on recombinant expression products.

The vector may be transfected into the cell by any means available to the skilled addressee. Preferably, the vector is introduced by calcium phosphate precipitation, electroporation, biolistics, lipofection, naked DNA, DEAE Dextran or adenoviral or retroviral infection. However, this invention is not restricted to these methods.

If the vector is to be introduced into a germ line to establish a transgenic line, then the transgene may be introduced using anyone of, but not limited to, i) pronuclear microinjection of DNA into a zygote; ii) transfection of preimplantation embryos with recombinant retroviruses carrying the gene of interest; iii) gene transfer into embryonic stem cells by using calcium phosphate-mediated DNA transformation, electroporation, retroviral infection or lipofection; iv) intracytoplasmic coinjection of unfertilized mouse oocytes with exogenous DNA and sperm heads whose membranes had been disrupted. Preferably, pronuclear microinjection is adopted.

The expression of the follistatin isoform may be increased or decreased to a level above the background level to modulate biological function. The degree of enhancement or reduction may be measured by the presence of isoform 315 or 288 protein, DNA, RNA, mRNA or bioactivity. Preferably, the expression of the transgene is measured by mRNA expression. From these measurements the relative levels of the isoforms can be adjusted. Modulation of a follistatin isoform to modulate expression and/or activity may be achieved by inducing expression of the follistatin isoform by transfection of a construct containing the follistatin isoform under the influence of a promoter or by overexpressing the gene in the cell. By introduction of an exogenous follistatin isoform or a construct to express an exogenous follistatin isoform, the ability of the follistatin isoform to modulate biological function may be achieved.

The cells are preferably transfected with the follistatin isoform by any means that introduces the follistatin isoform gene to the cell. Preferably, the gene encoding the follistatin isoform is transfected into the cell via an expression vector by methods routinely available to the skilled addressee or as described above.

Preferably a construct of a follistatin isoform is introduced or transfected into the cell to increase the expression the of follistatin isoform. Increasing the expression may be achieved by any means known to the skilled addressee including the induction of promoters in the construct. Vectors may be used with regulatory regions that respond to tetracycline, mifepristone or ecdysone.

However, the expression and/or activity may also be increased by indirect methods of targeting indirect regulators to upregulate the gene. These regulators may act on the promoters that cause expression of the gene or they may act on upstream or downstream molecules that affect the enzyme. For instance adrenodoxin and adrenodoxin reductase may be targeted because they are important for the flow of electrons. In transfection studies, fusion of all proteins into a single chimera, or transfected in tandem, may generate more enzymatic activity than addition of the genes alone. The chimera would be the molecule of choice for gene therapy work. It may be delivered in a vector with a promoter containing regulatory regions which preferably respond to metals, tetracycline, mifepristone or ecdysone. The chimera may also be delivered in tandem with a vector expressing another protein which may be essential for the expression of the biological function.

The expression of an individual isoform of follistatin may be regulated by molecules that regulate the splicing of the gene this might include small molecules designed for this purpose such as antisense molecules or RNAi or a small rationally designed molecule.

Regulation of the gene expression may generally be achieved by the use of molecules reacting with the promoter of the gene or with a promoter of a nuclear factor regulating the gene, or by RNA processing including splicing and degradation. The activity of proteins themselves may also be targeted by phosphorylation, or allosteric regulation or regulation of the protein degradation such as by the use of protease inhibitors.

Increased expression and/or activity of a follistatin isoform may be achieved by any means that can increase endogeneous follistatin isoform 315 expression and/or activity thereby resulting in the biological function.

In a related aspect of the present invention there is provided a cell characterised by modulated biological functioning, said cell comprising modulated expression and/or activity of follistatin isoform 315 and/or 288.

Preferably the cell is transfected with a gene encoding follistatin isoform 315 and/or 288. However, the cell may also have been exposed to compounds which modulated the endogeneous levels of isoform 315 and/or 288 such that endogeneous levels of isoform 315 and/or 288 were altered relative to background levels in the cell.

The cell may be any cell and may include, but is not limited to, cells of the reproductive system, urogenital system, endocrine system, neural system, digestive system, or hematopoietic system. Preferably the cells are cells of the testis, ovary, uterus, kidney, prostate, pituitary, pancreas, forebrain, cerebellum, spinal cord, salivary glands, liver, stomach, bone, thymus, heart, blood vessels, lung, muscle, skin and eye. More preferably they are vessel cells, blood cells, spermatocytes, oocytes, epithelial cells, skin cells, hair follicles, lung cells, hepatic cells, neural cells, islet cells, teeth cells, kidney, Sertoli cells, granulosa cells or germ cells

In yet another aspect of the present invention there is provided a transgenic animal characterised by modulated biological functioning and/or development, said animal comprising modulated expression and/or activity of follistatin isoform 315 and/or 288.

Preferably the animal is transfected with a gene encoding follistatin isoform 315 and/or 288. However, the animal may also have been exposed to compounds which can modulate endogeneous levels of isoform 315 and/or 288 such that the endogeneous levels of isoform 315 and/or 288 are altered relative to background levels in one or more of the cells of that animal.

The animal may be used for testing various compounds that can affect expression and/or activity of follistatin isoform 315 and/or 288 or an equivalent in an in vivo situation.

The animal may be any animal that can receive a construct that encodes the gene for follistatin, and the isoforms 315 and/or 288. Preferably the animal is a mammal. More preferably, the animal is a mouse or a rat.

Any method known to the skilled addressee that can generate a transgenic animal may be used. Preferably, the animal expresses the isoform 315 and/or 288.

The term “mammal” as used herein includes humans, primates, livestock animals (eg. horses, cattle, sheep, pigs, donkeys), laboratory test animals (eg. mice, rats, guinea pigs), companion animals (eg. dogs, cats) and captive wild animal (eg. kangaroos, deer, foxes).

As detailed hereinbefore, a further aspect of the present invention relates to the use of the invention in relation to the treatment and/or prophylaxis of disease conditions or other unwanted conditions.

The present invention therefore contemplates a method for therapeutically and/or prophylactically treating a development-related condition or a predisposition to the onset of a development related condition in a mammal, said method comprising modulating the functionally effective level of one or more follistatin isoforms, or derivative, fragment, homologue, mutant or variant thereof, in said mammal.

More particularly, the present invention therefore contemplates a method of therapeutically and/or prophylactically treating a development-related condition or a predisposition to the onset of a development related condition in a mammal, said method comprising modulating the functionally effective level of one or both of follistatin 315 or follistatin 288, or derivative, fragment, homologue, mutant or variant thereof, in said mammal.

A “development-related condition” as used herein relates to those conditions involved in the growing stages or developmental stages of an organism. In particular, it is a condition characterised by inappropriate or deficient biological functioning or development. They may include any congenital heart, lung, kidney, prostate, gastrointestinal or liver defects as well as defects affecting development of the brain and reproductive systems. The conditions also encompass growing conditions, wound healing in the skin and any such conditions affecting the growth and survival of an organism. Preferably the development-related condition is a lung, muscle, liver, eye, kidney, prostate or skin development-related condition and the functioning of isoforms 315 and/or 288 affects the development of these organs in the subject mammal.

More preferably, the subject condition is one characterised by aberrant:

(i) cellular or animal growth and survival;

(ii) capacity for movement (in the context of an animal);

(iii) skin and hair follicle development;

(iv) fetal development;

(v) embryogenesis; or

(vi) development of genital tubercles.

Still more preferably the subject biological functioning and/or development are upregulated by upregulation in the level of follistatin 315 and, optionally, the concurrent downregulation in the level of follistatin 288.

In another preferred embodiment, the condition is one characterised by aberrant vasculogenesis or angiogenesis. Still more preferably, the subject vasculogenesis or angiogenesis are inadequate and are upregulated by the upregulation in the level of follistatin 288 and, optionally, the concurrent downregulation in the level of follistatin 315. In another preferred embodiment, it is sought to downregulate vasculogenesis or angiogenesis, such as in the case of treating neoplastic conditions, by downregulating the level of follistatin 288 and, optionally, concurrently upregulating the level of follistatin 315.

These therapeutic and prophylactic aspects of the present invention are preferably achieved by administering an effective amount of a modulatory agent, as hereinbefore defined, for a time and under conditions sufficient to appropriately modulate one or more aspects of the development of the mammal.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the modulatory agent together with other proteinaceous or non-proteinaceous molecules which may facilitate the desired therapeutic or prophylactic outcome. For example, one may combine the method of the present invention with radiotherapy or chemotherapy if the treatment of neoplasias is being pursued via downregulation of angiogenesis in and around the tumour.

Administration of molecules of the present invention hereinbefore described [herein collectively referred to as “modulatory agent”], in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), respiratory, transdermal, intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, transdermally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip, patch and implant. Preferably, said means of administration is inhalation with respect to the treatment of airway inflammation and intravenously, intramuscularly or transdermally for other conditions.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, the subject agent may be administered together with an agonistic agent in order to enhance its effects. By “sequential” administration is meant a time difference of from seconds, minutes, hours or days between the administration of the two types of molecules. These molecules may be administered in any order.

The present invention also encompasses gene therapy whereby a gene encoding follistatin isoform 315 and/or 288 is regulated in a patient. Various methods of transferring or delivering DNA to cells for expression of the gene product protein, otherwise referred to as gene therapy, are disclosed in Gene Transfer into Mammalian Somatic Cells in vivo, N. Yang, Grit. Rev. Biotechn. 12(4): 335-356 (1992), which is hereby incorporated by reference.

Strategies for treating these medical problems with gene therapy include therapeutic strategies such as identifying a defective gene or protein and then adding a functional gene to either replace the function of the defective gene or to augment a slightly functional gene; or prophylactic strategies, such as adding a gene for the product protein that will treat the condition or that will make the tissue or organ more susceptible to a treatment regimen. As an example of a prophylactic strategy, a gene such as that for follistatin isoform 315 and/or 288 regulator may be placed in a patient and thus prevent occurrence of a development-related condition; or a gene that makes a cell more susceptible to other regulating factors in the cell or its environment to result in the desired biological function.

Many protocols for transfer of follistatin isoform 315 and/or 288 regulatory sequences are envisioned in this invention. Transfection of promoter sequences, or other sequences which would modulate the expression and/or activity of follistatin isoform 315 and/or 288 are also envisioned as methods of gene therapy. An example of this technology is found in Transkaryotic Therapies, Inc., of Cambridge, Mass., using homologous recombination to insert a “genetic switch” that turns on an erythropoietin gene in cells. See Genetic Engineering News, Apr. 15, 1994. Such “genetic switches” could be used to activate follistatin isoform 315 and/or 288 (or follistatin isoform 315 and/or 288 or an equivalent regulators in a cell.

Gene transfer methods for gene therapy fall into three broad categories: physical (e.g., electroporation, direct gene transfer and particle bombardment), chemical (lipid-based carriers, or other non-viral vectors) and biological (virus-derived vector and receptor uptake). For example, non-viral vectors may be used which include liposomes coated with DNA. Such liposome/DNA complexes may be directly injected intravenously into the patient. Additionally, vectors or the “naked” DNA of the gene may be directly injected into the desired organ, tissue or tumor for targeted delivery of the therapeutic DNA.

Gene therapy methodologies can also be described by delivery site. Fundamental ways to deliver genes include ex vivo gene transfer, in vivo gene transfer, and in vitro gene transfer.

Chemical methods of gene therapy may involve a lipid based compound, not necessarily a liposome, to ferry the DNA across the cell membrane. Lipofectins or cytofectins, lipid-based positive ions that bind to negatively charged DNA, may be used to cross the cell membrane and provide the DNA into the interior of the cell. Another chemical method may include receptor-based endocytosis, which involves binding a specific ligand to a cell surface receptor and enveloping and transporting it across the cell membrane.

Many gene therapy methodologies employ viral vectors such as retrovirus vectors to insert genes into cells. A viral vector can be delivered directly to the in vivo site, by a catheter for example, thus allowing only certain areas to be infected by the virus, and providing long-term, site specific gene expression. In vivo gene transfer using retrovirus vectors has also been demonstrated in mammary tissue and hepatic tissue by injection of the altered virus into blood vessels leading to the organs.

Viral vectors may be selected from the group including, but are not limited to, retroviruses, other RNA viruses such as poliovirus or Sindbis virus, adenovirus, adeno-associated virus, herpes viruses, SV 40, vaccinia and other DNA viruses. Replication-defective murine retroviral vectors are the most widely utilized gene transfer vectors and are preferred. Adenoviral vectors may be delivered bound to an antibody that is in turn bound to collagen coated stents.

Mechanical methods of DNA delivery may be employed and include, but are not limited to, fusogenic lipid vesicles such as liposomes or other vesicles for membrane fusion, lipid particles of DNA incorporating cationic lipid such as lipofectin, polylysine-mediated transfer of DNA, direct injection of DNA, such as microinjection of DNA into germ or somatic cells, pneumatically delivered DNA-coated particles, such as the gold particles used in a “gene gun,” inorganic chemical approaches such as calcium phosphate transfection and plasmid DNA incorporated into polymer coated stents. Ligand-mediated gene therapy, may also be employed involving complexing the DNA with specific ligands to form ligand-DNA conjugates, to direct the DNA to a specific cell or tissue.

The DNA of the plasmid may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products.

Gene regulation of follistatin isoform 315 and/or 288 may be accomplished by administering compounds that bind follistatin isoform 315 and/or 288 gene, or control regions associated with the follistatin isoform 315 and/or 288 gene, or corresponding RNA transcript to modify the rate of transcription or translation. Additionally, cells transfected with a DNA sequence encoding follistatin isoform 315 and/or 288 regulator may be administered to a patient to provide an in vivo source of follistatin isoform 315 and/or 288 regulator. For example, cells may be transfected with a vector containing a nucleic acid sequence encoding follistatin isoform 315 and/or 288 regulator.

The term “vector” as used herein means a carrier that can contain or associate with specific nucleic acid sequences, which functions to transport the specific nucleic acid sequences into a cell. Examples of vectors include plasmids and infective microorganisms such as viruses, or non-viral vectors such as ligand-DNA conjugates, liposomes, lipid-DNA complexes. It may be desirable that a recombinant DNA molecule comprising a follistatin isoform 315 and/or 288 regulator. DNA sequence is operatively linked to an expression control sequence to form an expression vector capable of follistatin isoform 315 and/or 288 regulator. The transfected cells may be cells derived from the patient's normal tissue, the patient's diseased tissue, or may be non-patient cells. For example, blood vessel cells removed from a patient can be transfected with a vector capable of expressing follistatin isoform 315 and/or 288 regulator of the present invention, and be re-introduced into the patient. The transfected cells demonstrate modulated follistatin isoform 315 and/or 288 expression and/or activity in the patient. Patients may be human or non-human animals. Cells may also be transfected by non-vector, or physical or chemical methods known in the art such as electroporation, incorporation, or via a “gene gun.” Additionally, follistatin isoform 315 and/or 288 regulator DNA may be directly injected, without the aid of a carrier, into a patient. In particular, follistatin isoform 315 and/or 288 regulator DNA may be injected into blood.

The gene therapy protocol for transfecting follistatin isoform 315 and/or 288 regulator into a patient may either be through integration of follistatin isoform 315 and/or 288 regulator DNA into the genome of the cells, into minichromosomes or as a separate replicating or non-replicating DNA construct in the cytoplasm or nucleoplasm of the cell. Modulation of follistatin isoform 315 and/or 288 expression and/or activity may continue for a long-period of time or may be reinjected periodically to maintain a desired level of follistatin isoform 315 and/or 288 expression and/or activity in the cell, the tissue or organ.

The modulated cells are intended to replace existing cells such that the existing development biology or biological function of the cells is modulated or the modulated cells may be used to infiltrate existing regions of disease to halt progression of the disease. Preferably, the expression and/or activity of follistatin isoform 315 and/or 288 is modulated by transfection of a gene encoding follistatin isoform 315 and/or 288 to the cells. The gene may then be over expressed or turned on to increase expression of the follistatin isoform 315 and/or 288 or it may be underexpressed to modify the levels of isoform 315 and/or 288 in the cell.

The replaced cells may be any cell that is affected by follistatin or may be tissue specific for the condition to be treated. Preferably the cell is selected from the group including, but not limited to, cells of the reproductive system, urogenital system, endocrine system, neural system, digestive system, or hematopoietic system. Preferably the cells are cells of the testis, ovary, uterus, kidney, prostate, pituitary, pancreas, forebrain, cerebellum, spinal cord, salivary glands, liver, stomach, bone, thymus, heart, blood vessels, lung, muscle, skin and eye. The cell may be a stem cell or an ES cell. More preferably they are vessel cells, blood cells, spermatocytes, oocytes, epithelial cells, skin cells, hair follicles, lung cells, hepatic cells, neural cells, islet cells, kidney, Sertoli cells, granulosa cells, germ cells. Preferably, the stem cells are capable of differentiating to any of the cells selected from the group including liver, lung, gastrointestinal, heart, ovary, or skin cells.

It is preferred that when using stem cells the cells may be transfected with a gene that encodes follistatin isoform 315 and/or 288, such that upon implantation to a tissue requiring treatment, the cells may differentiate to the cells of the region. Regulators modulating sequences of a vector that has been introduced to the cell can switch expression of follistatin isoform 315 and/or 288 on or off accordingly. These cells will eventually replace diseased cells.

Yet another aspect of the present invention relates to the use of an agent capable of modulating the functionally effective level of one or more follistatin isoforms, or derivative, fragment, homologue, mutant or variant thereof, in the manufacture of a medicament for the therapeutic and/or prophylactic treatment of a development-related condition, or a predisposition to the onset of a development related condition in a mammal.

Still a further aspect of the present invention relates to the use of an agent capable of modulating the functionally effective level of one or both of follistatin 315 or follistatin 288 or derivative, fragment, homologue, mutant or variant thereof, in the manufacture of a medicament for the therapeutic and/or prophylactic treatment of a development-related condition, or a predisposition to the onset of a development related condition in a mammal.

Preferably, said condition is one characterised by aberrant:

(i) cellular or animal growth and survival;

(ii) capacity for movement (in the context of an animal);

(iii) skin and hair follicle development;

(iv) fetal development;

(v) embryogenesis; or

(vi) development of genital tubercles.

Still more preferably the subject biological functioning and/or development are upregulated by upregulation in the level of follistatin 315 and, optionally, the concurrent downregulation in the level of follistatin 288.

In another preferred embodiment, the condition is one characterised by aberrant vasculogenesis or angiogenesis. Still more preferably, the subject vasculogenesis or angiogenesis are inadequate and are upregulated by the upregulation in the level of follistatin 288 and, optionally, the concurrent downregulation in the level of follistatin 315. In another preferred embodiment, it is sought to downregulate vasculogenesis or angiogenesis, such as in the case of treating neoplastic conditions, by downregulating the level of follistatin 288 and, optionally, concurrently upregulating the level of follistatin 315.

In yet another further aspect, the present invention contemplates a pharmaceutical composition comprising the modulatory agent as hereinbefore defined together with one or more pharmaceutically acceptable carriers and/or diluents. Said agents are referred to as the active ingredients.

A pharmaceutically acceptable carrier may be any carrier known to the skilled addressee which is not toxic to the patient and which can be admixed to form a pharmaceutical.

The active ingredients are preferably in a form that can be administered to enter the cell and affect the expression and/or activity of follistatin isoform 315 and/or 288.

The compositions of this invention can be administered to humans and other animals orally, rectally, parenterally (i.e. intravenously, intramuscularly, or sub-cutaneously), intracisternally, intravaginally, intraperitoneally, topically (as by powders, ointments, or drops), transdermally, bucally, or as an oral or nasal spray.

Compositions of this invention for parenteral injection comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

If desired, and for more effective distribution, the compounds can be incorporated into slow release or targeted delivery systems such as polymer matrices, liposomes, and microspheres.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

If desired, and for more effective distribution, the compounds can be incorporated into slow release or targeted delivery systems such as polymer matrices, liposomes, and microspheres.

The active compounds can also be in microencapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminium metahydroxide, bentonite, agar-agar, and tragacanth, and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Dosage forms for topical administration of a compound of this invention include powders, sprays, ointments and inhalants. The active compound is mixed under sterile conditions with a pharmaceutically acceptable carrier and any needed preservatives, buffers, or propellants which may be required.

### EXAMPLE 1

### Functional Analysis of 25 kb and 95 kb Genomic Sequences of the Human Follistatin Locus in the Follistatin Knockout Mice

In an attempt to define the human follistatin locus, the genomic constructs of the human follistatin gene, PAC-FS and pNEB-FS that contained 95 kb and 25 kb of genomic sequences, respectively, were used to create two transgenic mouse models. The aim was to drive the human follistatin gene within the constructs by the natural regulatory elements located within the genomic sequences of the human follistatin locus. The transgenic mice were then crossed with follistatin mutant heterozygotes (fs +/−) to assess whether the transgenes would be sufficiently active in the follistatin knockout background to rescue the follistatin knockout (fs −/−).

Materials and Methods

Characterization and Assembly of PCAC-FS and pNEB-FS

PAC-FS is the vector of P1 artificial chromosome (PAC) harboring a human follistatin genomic sequence that includes the follistatin gene and approximately 45 kb upstream and downstream sequences around the gene.

pNEB-FS is the vector which carries the human follistatin gene and its approximately 16 kb upstream and 3 kb downstream sequences in a 2.7 kb vector, pNEB 193 (New England Biolabs, Inc.).

The PAC-FS vector was characterized and the useful restriction sites in it were identified. Then a fragment of DNA, including the human follistatin gene cut from PAC-FS, was cloned into the pNEB 193 vector by several steps of DNA cloning, leading to pNEB-FS. pNEB-FS was further validated by restriction digestion. Following that, the DNA of PAC-FS and pNEB-FS was prepared for microinjection.

The sequence of human follistatin gene (about 6 kb) was published (Shimasaki, et al., 1988, Primary structure of the human follistatin precursor and its genomic organization Proceedings of the National Academy of Sciences of the United States of America 85 4218-4222). To map the restriction sites of the upstream and downstream sequences of the PAC-FS, several restriction endonucleases were used to cut the PAC-FS, then the DNA digests were electrophoresed in the agarose gel. The DNA in the gel was subsequently transferred onto two positively charged nylon membranes, which covered both sides of the gel via capillary transfer. Two different probes were separately used for Southern hybridization on the two positively charged nylon membranes. The two probes were made to sequences of exon 1 and exon 5 of the human follistatin gene, so they could detect the fragments which included exon 1 and exon 5, respectively. After autoradiography, the fragments containing exon 1 or exon 5 could be identified.

Based on results from using different combinations of restriction endonucleases, the location of specific restriction sites of the PAC-FS could be deduced.

Many restriction endonucleases, including Notl, EcoRl, Sall, Sse 83871, Swal, Fsel, Pacl, Sfil, Ascl, Xhol, Pvull, Kpnl, Spel, Ncol, Rsrll, Scal, Xmal, Aflll, Nhel, Nael, Sacl, Sacll, Hindlll, Ahdl, EcoRV, Pmel, Xbal, were used to characterize PAC-FS. The sites Sacl and Pacl were chosen for use in constructing the shorter genomic construct, pNEB-FS.

Because the sites Sacl and Pacl are unique within pNEB 193, this vector was chosen for the assembly of a shorter transgene that includes the follistatin gene and its approximately 16 kb upstream and 3 kb downstream flanking sequences.

PAC-FS and pNEB 193 were cut with the double digestion of Sacl plus Pacl. This released a fragment from PAC-FS which was then cloned into pNEB 193, designated as pNEB-S2. Similarly, PAC-FS and pNEB 193 were digested with Sacl. The released fragment from PAC-FS was then cloned into pNEB 193, designated as pNEB-S1. Subsequently, pNEB-S1 and pNEB-S2 were digested with Sacl. The released fragment from pNEB-S1 was cloned into the linearized NEB-S2 to form pNEB-FS. A PCR screen, designed to identify clones in the desired orientation, was used as a final screening step.

Microinjection of PAC-FS and pNEB-FS into Knock-Out Mice

(i) Experimental Animals

FVB mice were used for microinjection. FVB transgenic mice carrying the construct PAC-FS or pNEB-FS were crossed with a mixed C57/129 background mice heterozygous for the deleted follistatin allele (Matzuk et al., Nature 374 360-363, 1995) in an attempt to obtain the human follistatin transgenic mice in the mouse follistatin knockout background.

(ii) Pronuclear Microinjection of Transgenic DNA

The preparation of the DNA for transgenes was conducted using DNA of the correct size. This was entrapped in agarose gel and excised following electrophoresis as mentioned above. The DNA was then recovered from the gel with β-agarase (New England Biosciences). DNA was then mixed with ethidium bromide (10 mg/ml), before a phenol/chloroform extraction followed by treatment with water-saturated butanol. DNA was then precipitated and resuspended in 0.1×TE. As a final step, drop dialysis was used to remove excess salts and to then replace the solution with 0.1×TE. The methods associated with microinjection, i.e. hormonal stimulation and mating, embryo collection, DNA microinjection and embryo transfer, were conducted by methods familiar to those in the art.

(iii) Procedures for Genotyping

Genomic DNA was prepared from mouse ear-clips and then was used for genotyping. Genotyping to detect founders generated by pronuclear microinjection of zygotes was performed by PCR using four pairs of primers that target four different sites of transgenes. PCR primers were designed using primer3 (http://www-genome.wi.mit.edu/cgi-bin/primer/primer3_www.cgi) based on the human genomic sequence of the follistatin locus (source: NCBI, NT_{—}023081 contigs). The genotype of the transgenic mice born from the founders was determined by PCR reactions using one of these four pairs of primers. The sequences of these four pairs of primers, ‘10 kb-UP.AF & R’, ‘5 kb-UP.AF & R’, ‘Southern. AF & R’, and ‘1.5 kb-DOWN.AF & R’ are found in Table 2. The conditions for each PCR reaction were set up as described below.

To genotype the offspring from the cross-breeding program, genomic DNA was isolated from mouse tails. Three pairs of primers, human.FS.2F & R, mouse.FS.2F & Rand hHPRT.3F & R, were used to identify pups with different genetic backgrounds via PCR reactions. The details of these primers are found in Table 3.

The conditions of PCR reactions for these three pairs of primers were summarized as follows:

Reaction 1: using double pairs of primers
(human.FS.2F & Rand hHPRT.3F & R

| | | | |
|---|---|---|---|
| DNA 1 | | | |
| dNTPs | 2 mM | 3 | μI |
| 10x Buffer² | | 3 | μI |
| human.FS.2F | 50 μM | 0.4 | μI |
| human.FS.2R | 50 μM | 0.4 | μI |
| hHPRT.3F | 50 μM | 0.4 | μI |
| hHPRT.3R | 50 μM | 0.4 | μI |
| Tag DNA polymerase | 5 U/μI | 0.2 | μI |
| dH₂O | | to 30 | μI |

Reaction 2: using one pair of primers (mouse.FS.2F & R)

| | | | |
|---|---|---|---|
| DNA¹ | | | |
| dNTPs | 2 mM | 3 | μI |
| 10xBuffer² | | 3 | μI |
| mouse.FS.2F | 50 μM | 0.4 | μI |
| mouse.FS.2R | 50 μM | 0.4 | μI |
| Tag DNA polymerase | 5 U/μI | 0.2 | μI |
| dH₂O | | to 30 | μI |

¹10-100 ng genomic DNA was used. ²The buffer as supplied contained 500 mM KCl, 15 mM MgCl₂, 100 mM Tris-HCl (pH 9.0 at room temperature).

PCR conditions for both reactions 1 and 2 were set up as follows:

| | | | | |
|---|---|---|---|---|
| Initial denaturation | | | 94 C. | 5 min |
| Amplification: denaturation | | | 94 C. | 30 sec |
| annealing | {close oversize brace} | 30 cycles | 58 C. | 30 sec |
| extension | | | 72 C. | 30 sec |
| Final extension | | | 72 C. | 1 min |
| Holding temperature | | | 15 C. | Hold |

(iv) Establishment and Gross Examination of Independent Transgenic Lines

To maintain the transgenic lines and to further use the transgenic lines to cross with the follistatin knockout heterozygotes, the mating of founders with wild type mice was set up. Weights at birth and weaning times were recorded for 3 litters of each line. Gross examination was performed including appearance, movement and feeding. Fertility was also checked, based on whether there were offspring from the mating of transgenic mice with wild-type mice.

(v) Two Step Cross-Breeding Program to Obtain “Rescued Mice”

The transgenic mice carrying the human follistatin transgenes were crossed with the follistatin knockout heterozygotes via two steps of cross-breeding to generate “rescued” mice since the follistatin null mutants die soon after birth: rescued mice represent the mice that expressed the human follistatin transgenes, in the absence of the endogenous mouse gene.

(vi) General Characterization of Day 0 Pups from the Second Step of Cross-Breeding

Newborn pups were generally characterized by their appearance, weight, crown-rump length (CRL), breathing ability and survival rates. CRL was a length measured from the top of the head to the base of the tail.

(vii) Quantification of Human Follistatin mRNA Expression in Rescued Mice

Preparation of total RNA and mRNA from tissues is familiar to those skilled in the art.

In order to determine the mRNA expression levels of the human follistatin transgenes and the mouse follistatin endogenous gene in day 0 pups, reverse transcription was performed using Superscript II (Invitrogen) using mRNA from different organs (heart, lung, liver, kidney, muscle and skin).

Quantitative PCR for cDNA was then carried out with a real-time fluorimetric capillary based thermocycler (LightCycler™, Roche Diagnostic Co, Mannheim, Germany). The three pairs of PCR primers are described in Table 4 among which human.screen.2F & R was specific for human follistatin cDNA, mouse.screen.2F & R was specific for mouse follistatin cDNA, and β-actin.F & R was specific for mouse housekeeping gene β actin cDNA. They were designed to the human follistatin mRNA sequence (GENBANK: NM_{—}013409), the mouse follistatin mRNA sequence (GENBANK: NM_{—}008046) and the mouse β actin mRNA sequence (Ensembi Gene Report: ENSMUST00000031564), respectively. The real-time PCR conditions for these three pairs of primers were described in Table 5.

(viii) Experimental Design

The vectors pNEB-FS and PAC-FS that harbor the 25 kb and 95 kb genomic 5 sequences, respectively, were injected into the male pronuclei of mouse zygotes to generate transgenic mice. Subsequently, independent transgenic lines were established that were further crossed onto the mouse follistatin knockout background via the two-step cross-breeding with follistatin knockout heterozygotes. Consequently, from the second step of cross-breeding the rescued mice, knockout mice and wild-type mice can be obtained and phenotyped. The analyses included: functional analysis to see if the rescued mice were able to survive; morphological analysis including gross anatomical and histological examinations; quantification of follistatin mRNA expression in the rescued mice.

The following different mouse lines were used:
wt: wild-type mice
fs-ko: follistatin knockout mice
FS²⁵-wt: mice carrying 25 kb human follistatin transgene in the mouse wild-type background.
FS⁹⁵-wt: mice carrying 95 kb human follistatin transgene in the mouse wild-type background.
FS²⁵-ko: mice carrying 25 kb human follistatin transgene in the mouse follistatin knockout background.
FS⁹⁵-ko: mice carrying 95 kb human follistatin transgene in the mouse follistatin knockout background.
   Results
   Pronuclear Microinjection Data

For the pNEB-FS vector that was linearized and contained a 25 kb genomic sequence of the human follistatin locus, 323 fertilized oocytes with two pronuclei were collected for micro injection. Following micro injection, 208 embryos that looked morphologically normal at the one-cell stage were transferred into the oviducts of the foster mice, resulting in the birth of 36 pups (Table 6).

Likewise, for the PAC-FS vector that was circular (Camper and Saunders, 2000) and contained a 95 kb genomic sequence of the human follistatin locus, 1353 zygotes with two pronuclei were collected for microinjection. Subsequently, 810 embryos at the one-cell stage that looked morphologically normal were transferred into the oviducts of the foster mice, resulting in the birth of 89 pups (Table 1.5).

Transgenic Founders and Gross Examination of Transgenic Lines

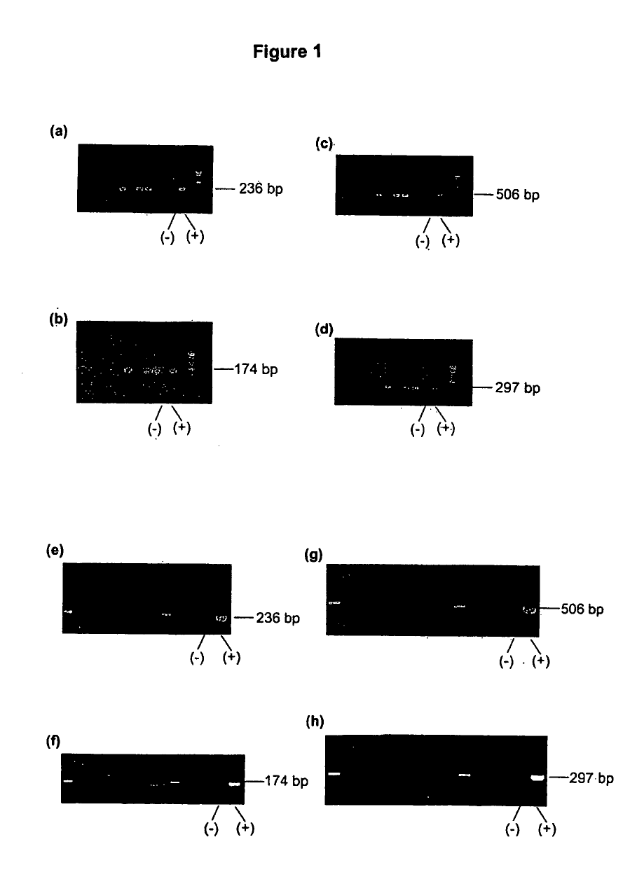

Three transgenic founders (1 female, 2 males), designated as FS²⁵-wt.1, FS²⁵-wt.2 and FS²⁵-wt.3, were obtained from pronuclear microinjection of the pNEB-FS vector; for the PAC-FS vector, two transgenic founders were obtained (2 females), designated as FS⁹⁵.wt.1 and FS⁹⁵.wt.2 (FIG. 1).

Transgenic mice of the lines FS⁹⁵-wt.1, FS⁹⁵-wt.2, FS²⁵-wt.1, FS²⁵-wt.2 and 5 FS²⁵-wt.3 were grossly normal in appearance, activity, fertility and weights.

The lines FS⁹⁵-wt.1, FS⁹⁵-wt.2, FS²⁵-wt.1 and FS²⁵-wt.2 were further analyzed for the mRNA expression of transgenes. The ratios in ovary, testis, muscle, kidney, liver and brain were approximately in the range of 0.05 to 0.0001 (Table 7).

General Characterizations of Day 0 Pups from the Second Step of Cross-Breeding

Two independent lines for each genomic construct that were further crossed 20 onto the mouse follistatin background.

Appearance of Pups:

The rescued mice FS²⁵-ko.1, FS²⁵-ko.2, FS⁹⁵-ko.1 and FS⁹⁵-ko.2 looked similar to fs-ko, except that the rescued mice appeared to be able to move more actively and breathe better at the initial stage after birth. The rescued mice were able to move their extremities freely and change a supine position to a prone position easily, but were unable to crawl like wild-type mice, suggesting that they were physically weaker than their wild-type littermates.

Weights and Crown-Rump Lengths (CRLs):

Wild-type (wt) was significantly heavier at birth than all the other lines. fs-ko was lighter than FS⁹⁵-ko.1 and wt mice (p<0.05) at birth.

The crown-rump lengths (CRLs) of wt were significantly longer than that of all other lines (p<0.05). When compared to the four lines of rescued mice, CRL of fs-ko was shorter than that of the two FS⁹⁵-ko lines (p<0.05) but not different from that of two FS²⁵-ko lines. In addition, there was no consistent difference in the CRLs between FS²⁵-ko and FS⁹⁵-ko lines although FS⁹⁵-ko.1 had a significantly longer CRL than FS²⁵-ko.2 (p<0.05).

Survival Rates and Breathing Activities:

There were distinctive differences in survival rates and breathing activities for the rescued mice and fs-ko within one day after birth. In the first observation after birth, the survival rates for fs-ko, FS²⁵-ko.1, FS²⁵-ko.2, FS⁹⁵-ko.1, FS⁹⁵-ko.2 and wt were 57.9%, 100%, 90%, 100%, 100% and 100%, respectively. Further, the second observation after birth, the survival rates for fs-ko, FS²⁵-ko.1, FS²⁵-ko.2, FS⁹⁵-ko.1, FS⁹⁵-ko.2 and wt were 10.5%, 90.9%, 80%, 90%, 88.9% and 100%, respectively. Both sets of data for fs-ko were significantly lower compared to either the four lines of the rescued mice or wt. However, on the second day after birth, neither the rescued mice nor fs-ko survived.

15.8% of fs-ko revealed good breathing, 36.8% of them were fair and 47.4% of them were bad at birth. In contrast, most of the rescued mice and wt displayed good breathing activities at birth.

Skin and Whisker Characteristics:

The skin of all four lines of the rescued mice looked taut and shiny, similar to that of fs-ko. The skin of some of the rescued mice appeared looser and less shiny when compared to fs-ko. There appeared to be no difference in whiskers between the rescued mice and fs-ko, which assumed a disoriented pattern.

Characteristics of External Genitalia:

In normal wt mice at birth, there is an obvious protrusion, called the genital tubercle, on the external genitalia. In contrast, the area where the external genitalia should form was completely flat in fs-ko. Most of the pups from all four lines of the rescued mice had a similar phenotype although some of them displayed a slight swelling in the external genital area. Obstruction of the lower urinary system was ruled out, since drops of urine could be pushed out with gentle pressure on the urinary bladders of the pups.

Histological Analysis of Day 0 Pups from the Second Step of Cross-Breeding

Liver:

Multiple comparisons of the weights of livers of fs-ko, wt, FS²⁵-ko.1, FS²⁵-ko.2, FS⁹⁵-ko.1 and FS⁹⁵-ko.2 revealed that at birth, the livers of fs-ko were significantly lighter than that of wt. Furthermore, the livers of wt were markedly heavier than that of fs-ko, FS²⁵-ko.1 and FS²⁵-ko.2. Although the weights of the livers of FS²⁵-ko.1 and FS²⁵-ko.2 were closer to that of fs-ko, and the weights of the livers of FS⁹⁵-ko.1 and FS⁹⁵-ko.2 were closer to that of wt.

GI (Gastrointestinal System):

The weights of GI, including stomach and bowels, of wt mice were significantly greater than those of fs-ko, FS²⁵-ko.1, FS²⁵-ko.2, FS⁹⁵-ko.1 and FS⁹⁵-ko.2.

Lung

The phenotypes of the lungs of the rescued mice were closer to that of wt than that of fs-ko, despite no differences between the rescued mice, except that the lungs of FS²⁵-ko.1 were more like that of fs-ko. The main differences between fs-ko and the rescued mice were the extent of the branching of bronchioles to form alveolar spaces and the thickness of the interalveolar septa between bronchioles. In the lung of fs-ko, the bronchioles appeared to branch less extensively and the interalveolar septa were also thicker compared to that of the rescued mice.

Heart:

Multiple comparisons of the weights of hearts of fs-ko, wt, FS²⁵-ko.1, FS²⁵-ko.2, FS⁹⁵-ko.1 and FS⁹⁵-ko.2 revealed that the hearts of wt were heavier than that of fs-ko. The weights of the hearts of the rescued mice were between that of fs-ko and wt, and were not significantly different from that of fs-ko or wt.

Skin:

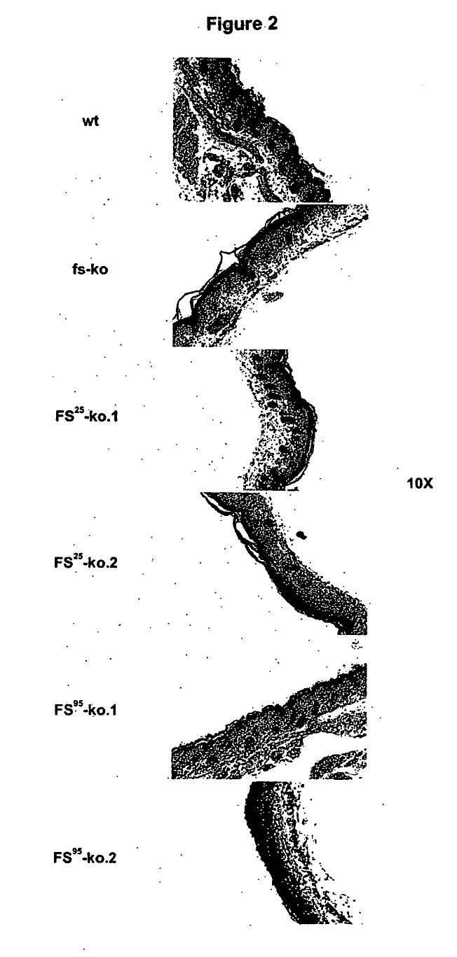

In the skin of fs-ko, most of the whole layer of the epidermis was thinner than that of wt (FIG. 2). In addition, the epidermis of wt demonstrated a wavy contour, in contrast to that of the fs-ko which tended to be flatter. Moreover, the numbers of hair follicles in the skin of fs-ko seemed to be less than that of wt (FIG. 2). For all these characteristics of skin described above, the four lines of the rescued mice displayed appearances that were intermediate between the fs-ko and the wt, but tended to be more like that of fs-ko. There also appeared to be no differences between four lines of the rescued mice. These histological characteristics appear to be compatible to the gross appearances of the rescued mice where they still displayed shiny and taut skin, but which was looser and less shiny than the fs-ko skin.

This work has identified pathological changes in the lungs of fs-ko mice which have not been described earlier. These changes, when added to the inability of these mice to expand their thoracic cavity due to inadequate skeletal muscle function, may represent the causes of death in these mice. The pattern observed histologically may indicate inadequate branching of the developing bronchial tree, leading to a decrease in lung mass and a limited alveolar space. Activin and follistatin have been shown to influence branching in several sites. Activin inhibits and follistatin stimulates branching in kidney and prostate (Cancilla, et al., Developmental Biology 237 145-158, 2001; Maeshima, et al., Cytokine Growth Factor Rev 12 289-298, 2001). The unopposed action of activin in the fs-ko mice could influence lung development. Detailed quantitative histological studies are required to confirm these qualitative observations. As indicated in the earlier discussion, the increased lung weights in the FS⁹⁵-ko mice may contribute to their extended neonatal survival.

There are significantly decreased numbers of oocytes in the perinatal ovaries in the fs-ko and four lines of the rescued mice. In some ovaries of fs-ko it was rare to see oocytes. Both human follistatin transgenes failed to rescue the ovarian phenotype of fs-ko, although the numbers of oocytes of the rescued mice appeared to be more than that of fs-ko.

Agenesis or severe under-development of the genital tubercle was found in fs-ko and all four lines of the rescued mice.

In conclusion, observation of the phenotypes of fs-ko and the rescued mice provide new insights into the biology of follistatin. Both the 25 kb and 95 kb human genomic transgenes could

### EXAMPLE 2

### Characterization of the Phenotype of the Human Follistatin Isoform-Specific Mouse Model

This example establishes transgenic animal models in which each of the follistatin isoforms 315 and 288 was specifically expressed. Constructs were created to express human follistatin-288 or follistatin-315 driven by the regulatory elements located in the 95 kb genomic sequence of the human follistatin locus. The transgene constructs, PAC-FSm1 (for follistatin-288) and PAC-FSm2 (for follistatin-315) were used to establish transgenic lines. Further, these transgenic mouse lines were crossed onto the mouse follistatin knockout background to generate “rescued mice” in which both follistatin isoforms have been separated. Subsequently, the distinct functions of both isoforms can be studied using these isoform-specific mouse models.

Expressing the Follistatin Isoforms 315 and 288

The DNA engineering in the follistatin gene was attempted to delete the intron 5 for the follistatin-288 specific construct and to delete intron 5 plus exon 6a for the follistatin-315 specific construct. The basis of this design was to force the translation of either follistatin-288 or follistatin-315.

Initially, two ways were considered to accomplish the DNA engineering in the follistatin gene. One is using the ‘knock-in’ technique to delete intron 5 or intron 5 plus exon 6a for follistatin-288 or follistatin-315, respectively, in the mouse genome; thereby the created homozygous knock-in mice would have only one of two isoforms. Alternatively, a transgene could be generated using the human follistatin locus. However, the human genomic sequence needs to be constructed by the technique of DNA engineering to generate two kinds of constructs of transgenes that were expected to be able to produce follistatin-288 or follistatin-315 in vivo. Taking advantage of the follistatin knockout mice that were already created by Matzuk's group (Matzuk, et al., 1995, supra), the mice carrying the isoform-specific transgenes in the mouse follistatin knockout background can be created after two steps of cross-breeding these transgenic mice with heterozygous follistatin knockout mice (using the heterozygous mutants is because homozygous follistatin mutants died soon after birth).

Construction of human follistatin isoform-specific transgenes in PAC-FS: PAC-FSm1 and PAC-FSm2

(a) Cloning m1 and m2 into the pCRII Vector

RT-PCR for m1 and m2:

One μg of human testis total RNA (BD Biosciences Clontech, CA, USA) was used for the first strand of cDNA synthesis of reverse transcription using RNase H⁻ reverse transcriptase, Superscript™II (Life Technologies). Two μl of the first strand reaction was used for the PCR reaction. The PCR reaction was run with the primers, fs-i-F and fs-i-R-1, and Taq DNA polymerase.

Cloning m1 and m2 into the pCRII Vector:

The PCR reaction was electrophoresed and the predicted products of PCR reactions, 396 bp and 660 bp, were excised from the low melting temperature (LMT) gel. The target bands were then cloned into the pCRII vector (Invitrogen) by T A cloning technique and in-gel ligation method. The new formed vectors were designated as pCR-m1 and pCR-m2 for the segments m1 and m2, respectively. The ligation reaction was further transformed into DH5α cells. The cells were then grown on LB agar with kanamycin at 37 C overnight. After that, PCR screening of colonies was performed with the primers, fs-i-F and fs-i-R-1. Six colonies for pCR-m1 and pCR-m2 were picked for further growing. Following that, glycerol stocks and DNA mini-preps of pCR-m1 and pCR-m2 were made. The DNA of three clones for both pCR-m1 and pCR-m2 was then sent to be sequenced. The clones with the correct sequences were kept for further experiments.

(b) Cloning S3 into the pNEB193 Vector

Acquisition of S3 from pNEB-S2:

pNEB-S2 and pNEB193 were cut with double digestion of EcoRI and Hindlll, releasing S3 from pNEB-S2 as well as linearizing and preparing pNEB193 for ligation with S3.

Ligation and Transformation:

The digestion reactions were then electrophoresed in agarose gel. The target bands, S3 (1.8 kb) and pNEB193 (2.7 kb), were entrapped in LMT gel. DNA extraction of the target bands was performed by digesting the LMT gel with β-agarase I (New England BioLabs). Subsequently, a ligation reaction was set up for S3 and linearized pNEB193 with the ratio of 1:1 between the insert and the vector at room temperature overnight. The newly formed vector was designated as pNEB-S3 The INVαF′ cells were grown on LB agar containing ampicillin at 37 C overnight, after being transformed by the ligation reaction. PCR screening of colonies was made with the primers, fol.5.F and fol.5.R. Several positive colonies were obtained. Two colonies were chosen for glycerol stocks and DNA mini-preps for further experiments.

(c) Cloning S4 into the pSL1180 Vector

Acquisition of S4 from pNEB-S2:

pNEB-S2 and pSL1180 were double digested with EcoRV and Ncol. This released S4 from pNEB-S2 and also linearized pSL1180.

Ligation and Transformation:

The target bands, S4 (2.6 kb) and linearized pSL1180 (3.4 kb), were obtained via electrophoresis of the digestion reaction mixtures. DNA extraction was then performed by QIAquick Gel Extraction kits (QIAGEN, Vic., Australia). Subsequently, a ligation reaction was set up for S4 and linearized pSL1180 with the ratio of 1:1 between the insert and the vector at 16 C overnight. The newly formed vector was designated as pSL-S4. The DH5α cells were grown on LB agar containing ampicillin at 37 C overnight, after being transformed by the ligation reaction. PCR screening of colonies was made with the primers, fs-x-F and fs-i-R-2. Several positive colonies were obtained. Three colonies were chosen for glycerol stocks and further DNA mini-prep.

(d) Assembly of pNEB-S3m1 and pNEB-S3m2

Restriction Digestion:

pCR-m1, pCR-m2 and pNEB-S3 were double digested with EcoRV and Xbal. This released m1 and m2 as well as cutting the segment intended to be modified from pNEB-S3. The digestion reactions were then electrophoresed. The target bands, 546 bp, 3.4 kb and 282 bp, were obtained in LMT gel.

Ligation, Transformation and PCR Screening:

Two ligation reactions (one for m1, the other for m2) were set up with T₄ DNA ligase to form the new vectors pNEB-S3 ml and pNEB-S3m2, respectively. Transfer of the ligation products into cells was done by transforming electrocompetent DH10B cells. Following that, DH10B cells were grown on LB agar containing ampicillin at 37 C overnight. Further, PCR screening for the desired colonies was made with the primers, fol.5.F and Fs-i-R-1. Several positive colonies were obtained. Two positive colonies were chosen from each of pNEB-S3m1 and pNEB-S3m2 groups to be grown up for glycerol stocks and further experiments.

(e) Assembly of pSL-S4m1 and pSL-S4m2

Restriction Digestion:

pNEB-S3m1, pNEB-S3m2 and pSL-S4 were double digested with EcoRV and PshAI. This released S3m1 and S3m2 from the vectors as well as cut off the segment intended to be modified from pSL-S4. The digestion reactions were then electrophoresed. The target bands, 707 bp, 4.8 kb and 443 bp, were obtained in LMT gel. DNA extraction from gel was made with QIAquick Gel Extraction kits (QIAGEN).

Ligation, Transformation and PCR Screening:

Two ligation reactions (one for m1, the other for m2) were set up with T₄ DNA ligase at cyclic temperature to form two new vectors pSL-S4 ml and pSL-S4m2, respectively. Transfer of the ligation products into cells was done by transforming electrocompetent DH10B cells. Following that, DH10B cells were grown on LB agar containing ampicillin at 37 C overnight. Further, PCR screening for the desired colonies was made with the primers, S4-M-F and fs-i-R-2. Several positive colonies were obtained. Two positive colonies were chosen from each of pSL-S4m1 and pSL-S4 m2 groups to be grown up for glycerol stocks and further experiments.

(f) Assembly of pNEB-S2m1 and pNEB-S2m2

Restriction Digestion:

pSL-S4m1, pSL-S4m2 and pNEB-S2 were double digested with EcoRV and Ncol. This released S4m1 and S4m2 from the vectors as well as cutting the segment intended to be modified from pNEB-S2 The digestion reactions were then electrophoresed. The target bands, 2.2 kb, 9.4 kb and 1.9 kb, were obtained in LMT gel DNA extraction from gel was made with β-agarase.

Ligation, Transformation and PCR Screening:

Two ligation reactions (one for m1, the other for m2) were set up with T₄ DNA ligase at cyclic temperature to form two new vectors pNEB-S2 ml and pNEB-S2m2, respectively. Transfer of the ligation products into cells was done by transforming electrocompetent DH10B cells. Following that, DH10B cells were grown on LB agar containing ampicillin at 37 C overnight. Further, PCR screening for the desired colonies was made with the primers, fol.5.F and fs-i-R-1. Several positive colonies were obtained. Two positive colonies were chosen from each of pNEB-S2m1 and pNEB-S2m2 groups to be grown up for glycerol stocks and further experiments.

(g) Assembly of pNEB-FSm1 and pNEB-FSm2

Restriction Digestion:

pNEB-S2m1, pNEB-S2m2 and pNEB-S1 were digested with the restriction endonuclease Sacl, resulting in the release of S1 from pNEB-S1 and the linearization of pNEB-S2m1, pNEB-S2m2. The digestion reactions were then (electrophoresed. The target bands that were 12 kb, 16 kb and 12 kb for pNEB-S2m1, S1 and pNEB-S2m2, respectively, were obtained in LMT gel. DNA extraction from the gel was achieved with β-agarase I.

Ligation, Transformation and PCR Screening:

Two ligation reactions (one for m1, the other for m2) were set up with T₄ DNA ligase at cyclic temperature to form two new vectors, pNEB-FSm1 and pNEB-FSm2. Transfer of the ligation products into cells was done by transforming electrocompetent DH10B cells. Following that, DH10B cells were grown on LB agar containing ampicillin at 37 C overnight. Further, PCR screening for the desired colonies was made with the primers, fol.1.F and fs-sac-R. Several positive colonies were obtained. Two positive colonies were chosen from each of pNEB-FSm1 and pNEB-FSm2 groups to be grown up for glycerol stocks and further experiments.

(h) Cloning S5 into the pNEB193 Vector

Acquisition of S5 from PAC-FS:

PAC-FS and pNEB193 were double digested with Ascl and BamHI, releasing S5 from PAC-FS as well as linearizing and preparing pNEB193 for ligation with S5.

Ligation and Transformation:

The digestion reactions were then electrophoresed in the agarose gel. The target bands, S5 (18 kb) and pNEB193 (3 kb), were entrapped in LMT gel DNA extraction of the target bands was performed using β-agarase. Subsequently, a ligation reaction was set up for S5 and linearized pNEB193 with the ratio of 1:1 between the insert and the vector and with T₄ DNA ligase at cyclic temperature to make a new vector, pNEB-S5. The DH5α cells were grown on LB agar containing ampicillin at 37 C overnight, following transformation by the ligation reaction. PCR screening of colonies was made with the primers, 1.5Down.AF and 1.5Down.AR. Several positive colonies were obtained. Two colonies were chosen for glycerol stocks and further DNA mini-preps.

(i) Assembly of pNEB-S5.m1 and pNEB-S5.m2

Restriction Digestion:

pNEB-FSm1, pNEB-FSm2 and pNEB-S5 were double digested with Asci and Spel. This released the segments from pNEB-FSm1 and pNEB-FSm2 that would be used to exchange the corresponding segment in S5 to get the isoform specific fragments extended to S5. At the same time, the digestion reaction cut off the segment intended to be modified from pNEB-S5 thereby allowing the linearized and digested pNEB-S5 to be available for use in the ligation reaction with the segments from pNEB-FSm1 and pNEB-FSm2. The digestion reactions were then electrophoresed. The target bands, 7 kb, 14 kb and 7 kb, for further ligation reactions were obtained in LMT gel. DNA extraction from the gel was made with β-agarase I.

Ligation, Transformation and PCR Screening:

Two sets of ligation reactions (one for m1, the other for m2) were performed with T₄ DNA ligase at 16 C overnight to make two new vectors, pNEB-S5.m1 and pNEB-S5.m2. Transfer of the ligation products into cells was done by transforming DH5α cells. Following that, DH5α cells were grown on LB agar containing ampicillin at 37 C overnight. Further, PCR screening for the desired colonies was made with the primers, fs-56-F and Fs-i-R-2. Several positive colonies were obtained. Three positive colonies were chosen from each of pNEB-S5 ml and pNEB-S5 m2 groups to be grown up for glycerol stocks and further experiments.

(j) Assembly of PAC-1/4Step

Restriction Digestion:

PAC-FS was cut with the restriction endonuclease Notl, resulting in the release of three fragments from PAC-FS. These three fragments were separated by pulsed field gel electrophoresis (PFGE). The two target bands that were 52 kb and 15 kb in size for further ligation were obtained in LMT gel.

Ligation, Transformation and PCR Screening:

The in-gel ligation reaction was set up with T₄ DNA ligase at 16 C overnight to make a new vector, PAC-1/4Step. Transfer of the ligation products into cells was done by transforming electrocompetent DH10B cells. Following that, DH10B cells were grown on LB agar containing kanamycin at 37 C overnight. Further, PCR screening for the desired colonies was made with the primers, prim-4steps-3F and prim-4steps-3R. One positive colony was obtained. The positive colony was then grown up for glycerol stocks and further experiments.

(k) Assembly of PA C-2/4Step.m1 and PA C-2/4Step.m2

Restriction Digestion:

pNEB-S5.m1, pNEB-S5.m2 and PAC-1/4Step were double digested with AatII and BamHI. This released the most parts of the modified S5 segments from pNEB-S5.m1 and pNEB-S5.m2 as well as the backbone of PAC vector from PAC-1/4Step. The digestion reactions were then electrophoresed by PFGE. The target bands that were 18 kb, 15 kb and 18 kb in size for further ligations were obtained in LMT gel.

Ligation, Transformation and PCR Screening:

Two in-gel ligation reactions (one for m1, the other for m2) were set up with T₄ DNA ligase at 16 C overnight to make two vectors, PAC-2/4Step.m1 and PAC-2/4Step.m2. Transfer of the ligation products into cells was done by transforming electrocompetent DH10B cells. Following that, DH10B cells were grown on LB agar containing kanamycin at 37 C overnight. Further, PCR screening for the desired colonies was made with the primers, prim-4steps-3F and BamHI-R. Several positive colonies were obtained. Four positive colonies were chosen from each of PAC-2/4Step.m1 and PAC-2/4Step.m2 groups to be grown up for glycerol stocks and further experiments.

(l) Assembly of PAC-3/4Step.m1 and PAC-3/4Step.m2

Restriction Digestion:

PAC-2/4Step.m1, PAC-2/4Step.m2 and PAC-1/4Step were cut with the restriction endonuclease BamHI. This linearized PAC-2/4Step.m1 and PAC-2/4Step.m2 as well as released a segment required for further ligation from PAC-1/4Step. The digestion reactions were then electrophoresed by PFGE. The 3 target bands that were 33.5 kb in size for further ligation reactions were obtained in LMT gel.

Ligation, Transformation and PCR Screening:

Two in-gel ligation reactions (one for m1, the other for m2) were set up with T₄ DNA ligase at 16 C overnight to make two new vectors, PAC-3/4Step.m1 and PAC-3/4Step.m2. Transfer of the ligation products into cells was done by transforming electrocompetent DH10B cells. Following that, DH10B cells were grown on LB agar containing kanamycin at 37 C overnight. Further, PCR screening for the desired colonies was made with the primers, prim-4steps-3F and prim-4steps-3R. Several positive colonies were obtained. Six positive colonies were chosen from each of the PAC-3/4Step.m1 and PAC-3/4Step.m2 groups to be grown up for glycerol stocks and further experiments.

(m) Assembly of PAC-FSm1 and PAC-FSm2

Restriction Digestion:

PAC-3/4Step.m1, PAC-3/4Step.m2 and PAC-FS were double digested with BsiWI and AatII. This cut off the backbone sequence of the PAC vector from PAC-3/4Step.m1 and PAC-3/4Step.m2 as well as releasing a segment required for the final ligation from PAC-FS. The digestion reactions were then electrophoresed by PFGE. The target bands that were 51 kb, 56 kb and 51 kb in size for further ligation reactions were obtained in LMT gel. DNA extraction from gel was made with β-agarase I.

Ligation, Transformation and PCR Screening:

Two ligation reactions (one for m1, the other for m2) were set up with T₄ DNA ligase at cyclic temperature (Section 2.2.2.7) to complete the final constructs, PAC-FSm1 and PAC-FSm2 (FIG. 7 0.17 k &1). Transfer of the ligation products into cells was done by transforming the electrocompetent DH10B cells. Following that, DH10B cells were grown on LB agar containing kanamycin at 37 C overnight. Further, PCR screening for the desired colonies was made with the primers, prim-4steps-3F and prim4steps-3R. Several positive colonies were obtained. Six positive colonies were chosen from each of PAC-FSm1 and PAC-FSm2 groups to be grown up for glycerol stocks and further experiments.

Microinjection of Isoforms 315 and/or 288

(i) Experimental Animals

FVB mice used for pronuclear microinjection were as in Example 1.

(ii) Pronuclear Microinjection of Transgenic DNA

The preparation of the DNA of transgenes, i.e. PAC-FSm1 and PAC-FSm2, for microinjection was conducted as follows. DNA was entrapped in agarose gel and excised following electrophoresis as mentioned above. The DNA was then recovered from the gel with β-agarase (New England Biosciences). DNA was then mixed with ethidium bromide (10 mg/ml), before a phenol/chloroform extraction followed by treatment with water-saturated butanol. DNA was then precipitated and resuspended in 0.1×TE. As a final step, drop dialysis was used to remove excess salts and to then replace the solution with 0.1×TE The methods associated with microinjection, are as described in Example 1.

(iii) Procedures for Genotyping

Genomic DNA was isolated from mouse tails and then was used for genotyping. Four pairs of PCR primers, ‘10 kb-UP.AF & R’, ‘5 kb-UP.AF & R’, ‘Southern.AF & R’, and ‘1.5 kb-DOWN.AF & R’, were used for detecting the founders produced by the pronuclear microinjection of zygotes. 10 kb-UP.AF & R and 5 kb-UP.AF & R were targeting the sites about 10 kb upstream and 5 kb upstream of the human follistatin gene, respectively. Southern.AF & R was targeting the site within the follistatin gene and 1.5 kb-DOWN.AF & R was targeting the site about 1.5 kb downstream of the follistatin gene. In addition, the characteristics of these four pairs of PCR primers can be referred to in Table 1.1.

For genotyping the offspring from cross-breeding, genomic DNA was isolated from mouse tails. Three pairs of primers, human.FS.2F & R, mouse.FS.2F & R and hHPR.3F & R, were used to identify pups with different genetic backgrounds via PCR reactions. Human.FS.2F & R was targeting the site within the human follistatin gene, whereas mouse.FS.2F & R was targeting the site within the mouse follistatin gene. hHPR.3F & R was targeting the site within the replacement cassette for the mouse follistatin gene in follistatin knockout mouse genome. Further, the characteristics of these primers were tabulated in Table 1.2. The conditions for the PCR reactions for these three pairs of primers were summarized in Example 1.

(iv) The Generation of Rescued Mice

These transgenic mice carrying the human follistatin transgenes were crossed with the follistatin knockout heterozygotes via two steps of cross-breeding, to generate “rescued” mice since the follistatin knockout homozygotes die soon after birth: rescued mice represent the mice that expressed the human follistatin transgenes, in the absence of the endogenous mouse gene.

(v) General Characterization

To maintain the transgenic lines and further use the transgenic lines to cross onto the follistatin knockout background, the mating of founders with wild-type mice (wt) was set up. Weights at birth and weaning time were recorded for 3 litters of each line. Gross examination was performed including assessment of their appearance, movement and eating behaviour. Fertility was also checked, based on whether there were offspring from the matings of transgenic mice with wt mice.

For the follistatin isoform-specific rescued mice, the newborn pups were generally characterized by their appearance, weights, crown-rump length (CRL), breathing ability and survival rates. CRL was a length measured from the top of the head to the base of the tail. If the rescued pups were able to survive, the observations would then continue.

(vi) Experimental Design

The introduction of transgenes into the mouse genome was performed by pronuclear microinjection. The transgenic founders were then mated with wt mice to establish independent transgenic lines. The transgenic mouse lines were subsequently crossed onto the mouse follistatin knockout background by two steps of cross-breeding to generate “rescued mice”. Initial characterization of the phenotypes of the follistatin isoform-specific models is presented in this chapter.

The following different mouse lines were established:
wt: wild-type mice
fs-ko: follistatin knockout mice
FS⁹⁵-288-wt: mice carrying 95 kb human follistatin transgenes which specifically express human follistatin-288 in the mouse wild-type background
FS⁹⁵-315-wt: mice carrying 95 kb human follistatin transgenes which specifically express human follistatin-315 in the mouse wild-type background
FS⁹⁵-288-ko: mice carrying 95 kb human follistatin transgenes which specifically express human follistatin-288 in the mouse follistatin knockout background
FS⁹⁵-315-ko: mice carrying 95 kb human follistatin transgenes which specifically express human follistatin-315 in the mouse follistatin knockout background.
   Results
   (i) Pronuclear Microinjection Data

For the PAC-FSm1 vector that was constructed in an attempt to express follistatin-288 only, as well as containing a 95 kb genomic sequence of the human follistatin locus and a 15 kb backbone sequence of the PAC vector, 359 eggs in total at the pronuclear stage were collected for microinjection. Following that, 211 embryos that looked healthy at the one-cell stage were transferred into the oviducts of the foster mice and 8 pups were born.

Similarly, for the PAC-FSm2 vector that was constructed in order to express follistatin-315 only, as well as containing a 95 kb genomic sequence of the human follistatin locus and a 15 kb backbone sequence of the PAC vector, 810 eggs in total were collected for microinjection. Subsequently, 545 embryos at the one-cell stage that looked healthy were transferred into the oviducts of the foster mice and 48 pups were born (Table 8).

(ii) Transgenic Founders

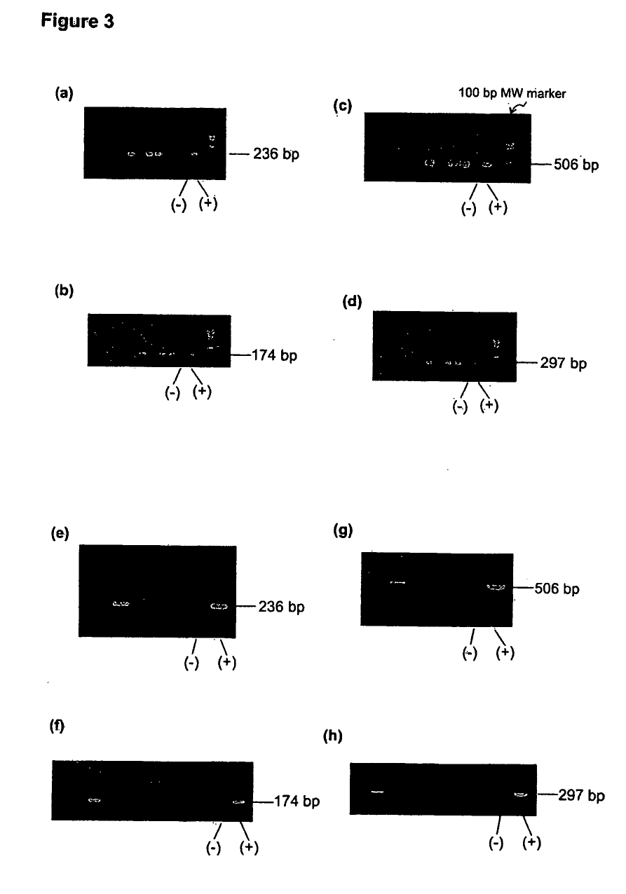

Three transgenic founders (2 females, 1 male), designated as FS⁹⁵-288-wt.1, FS⁹⁵-288-wt.2 and FS⁹⁵-288-wt.3, were obtained from the pronuclear microinjection of the PAC-FSm1 vector; for the PAC-FSm2 vector, one transgenic founder was obtained (female), designated as FS⁹⁵-315-wt.1 (Table 2.1, FIG. 3).

(iii) Transgenic Lines Carrying Transgenes in Wild-Type Background Appear Normal

Independent transgenic lines were generated by crossing founders with wild-type FVB mice. The transgenic mice of the lines FS⁹⁵-288-wt.1, FS⁹⁵-288-wt.2, FS⁹⁵-288-wt.3 and FS⁹⁵-315-wt.1 were grossly normal in appearance, activity, weight at birth and at weaning.

(iv) Weights and Crown-Rump Length (CRL) of Follistatin Isoform-Specific Rescued Mice at Birth

Follistatin isoform-specific rescued mice are designated as follows:
i) Follistatin-288 specific rescued mice: FS⁹⁵-288-ko.1 and FS95-288-ko.2 represent the rescued mouse lines from the transgenic lines FS⁹⁵-288-wt.1 and FS⁹⁵-288-wt.2, respectively.
ii) Follistatin-315 specific rescued mice: FS⁹⁵-315-ko.1 represents the rescued mouse line from the transgenic line FS⁹⁵-315-wt.1.

The lines FS⁹⁵-288-ko.1 and FS⁹⁵-288-ko.2 were no different from fs-ko in weight and CRL at birth, while they were smaller than wt. In contrast, the line FS⁹⁵-315-ko.1 was bigger than fs-ko, FS⁹⁵-288-ko.1 and FS⁹⁵-288-ko.2 in weight and CRL at birth; at the same time, FS⁹⁵-315-ko.1 was similar to wt in both weight and CRL at birth (Table 9).

(v) Gross Examination of Follistatin Isoform-Specific Rescued Mice at Birth

From gross examination, FS⁹⁵-288-ko.1 and FS⁹⁵-288-ko.2 are similar to FS⁹⁵— ko.1 and FS⁹⁵-ko.2 (referred to Example 1). FS⁹⁵-ko carries the 95 kb genomic sequence (PAC-FS) of the human follistatin locus, which was used as a base to delete intron 5 to make a transgene construct (PAC-FSm1) for FS⁹⁵-288-ko. FS⁹⁵-288-ko displayed better respiratory function as indicated by their pink skin color, in contrast to fs-ko which always showed respiratory distress with purple skin color at birth. FS⁹⁵-288-ko was also more active and moved more freely than fs-ko. FS⁹⁵-288-ko lived for about 12 hours longer than FS-ko which usually died within 1-2 hours after birth. However, in other aspects, FS⁹⁵-288-ko was quite similar to fs-ko. They were much smaller than wt pups. They had disoriented whiskers, taut and shiny skin and a flatter urogenital tubercle. Further, no milk was observed in their stomachs.

FS⁹⁵-315-ko.1 looked quite different from FS⁹⁵-288-ko and fs-ko. Their sizes were similar to wt pups. Some of them still had slightly disoriented whiskers. Their skin looked normal, without the taut and shiny characteristics of the skin of the fs-ko. Further, the genital tubercle in FS⁹⁵-315-ko.1 was more obvious than that of the FS⁹⁵-288-ko and fs-ko mice, but was still flatter than that of the wt pups. The tails of FS⁹⁵-315-ko.1 were similar to wt pups in length at birth. Afterwards, the tips of the FS⁹⁵-315-ko.1 started to display a red color and became shorter than that of wt. Importantly, FS⁹⁵-315-ko.1 appeared as active as wt pups and milk could be easily identified in their stomachs.

(vi) Follistatin-315 Specific Genomic Transgene Prevented the Neonatal Fatality of Follistatin Knockout Mice in Contrast to Follistatin-288 Transgene

FS⁹⁵-315-ko.1 survived, in remarkable contrast to FS⁹⁵-288-ko that died around one day after birth. Some other interesting phenotypes became apparent as FS⁹⁵-315-ko.1 pups developed. First, they grew more slowly than wt pups. Their tails were shorter compared to their body size. Importantly, the tips of their tails were usually red and showed small nodules along the tip of the tail. The red color at the tip of their tails sometime became black in color, implying a gangrenous change due to compromised blood supply. At about 10 days of age, a parting of their hair appeared in the midline of the dorsum of their heads and backs but then disappeared by 3 weeks of age. The disoriented whisker pattern became less obvious with age. The penis and clitoris looked normal, although they seemed smaller than those of wt mice. Further, they had microphthalmia. FS⁹⁵-315-ko.1 males proved to be fertile and appeared to be able to live up to 2˜3 months. However, FS⁹⁵-315-ko.1 females appeared infertile although they did mate.

In summary, the human follistatin-315 specific transgene can prevent the neonatal death of FS-ko mice, while the human follistatin-288 specific transgene could not. This presents clear evidence that both follistatin isoforms have distinct functions in some aspects of follistatin biology.

The transgenes used to create both follistatin isoform-specific models were using a 95 kb genomic sequence of the human follistatin locus as a basis to perform the DNA engineering. The flanking regions of both isoform-specific transgenes are exactly the same. In other words, the natural regulatory elements within the transgene constructs that drive follistatin expression in vivo are the same in both the follistatin-288 specific and the follistatin-315 specific transgenes. Thus, in theory, FS⁹⁵-288-ko and FS⁹⁵-315-ko should have similar levels of follistatin and the difference is that only follistatin-288 is expressed in FS⁹⁵-288-ko and only follistatin-315 is expressed in FS⁹⁵-315-ko. The results presented clearly indicate that the FS⁹⁵-315-ko line can survive but not the FS⁹⁵-288-ko mice.

Follistatin-288 may have a much higher affinity to heparin sulfate proteoglycans present in plasma membranes of cells in comparison to follistatin-315, leading to the relatively easy diffusion for follistatin-315 to reach the target sites. Therefore, follistatin-315 was still able to efficiently exert most of its biological functions in FS⁹⁵-315-ko. This rescue occurs despite probable under-expression of follistatin both in FS⁹⁵-288-ko and in FS⁹⁵-315.

Also, FS⁹⁵-ko and FS⁹⁵-315-ko should express similar levels of follistatin. Further, FS⁹⁵-ko expresses both follistatin-288 and follistatin-315, the amounts of which would be 20 times the amount of follistatin-288 based on the published data (Michel, et al., 1990), so the levels of follistatin-315 in FS⁹⁵-ko should be quite similar to those in FS⁹⁵-315-ko. However, the phenotypes of FS⁹⁵-ko are so different from those of FS⁹⁵-315-ko. Without being limited by theory, it is considered that that follistatin-288 is, in some way, counter-acting the biological actions of follistatin-315.

FS⁹⁵-315-ko has overcome growth restriction that was revealed in fs-ko, FS²⁵-ko, FS⁹⁵-ko and FS⁹⁵-288-ko.

Follistatin-315 is also able to reverse the phenotype of shiny and taut skin from the effects of the follistatin knockout. In contrast, follistatin-288 appears not to be able to have this kind of effect based on the phenotypes of FS⁹⁵-288-ko. This indicates that both follistatin isoforms may have distinct biological roles in skin.

In FS⁹⁵-315-ko, their whiskers were less disoriented and their urogenital tubercles were more obvious compared to fs-ko or FS⁹⁵-288-ko. However, when the FS⁹⁵-315-ko pups grew up, these phenotypes became less obvious and were more like that of wt mice. Accordingly, follistatin-315 may partially rescue these two phenotypes of fs-ko at the initial stage and that follistatin may not be so important at the later stage.

The distinct phenotypes between FS⁹⁵-288-ko and FS⁹⁵-315-ko imply that both follistatin isoforms are different in some aspects of their actions.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

TABLE 2
PCR primers for detecting transgenic founders

| | | | | |
|---|---|---|---|---|
| | | Tm | | Product |
| Primer | Sequence (5′ 3′) | (C) | Targeting site | size |

| | | | | |
|---|---|---|---|---|
| 10kb-UP- | F:GTGGCCTTCTGGAGACTGAG | 60 | 10 kb upstream of | 236 bp |
| A.F | (SEQ ID NO:1) | | human follistatin | |
| | | | gene | |
| 10kb-UP- | R:CATGATGGCACGAACCTGTA | 60.5 | | |
| A.R | (SEQ ID NO:2) | | | |
| 5kb-UP- | F:AACATCCCCATTCTAAGGCA | 59.3 | 5 kb upstream of | 174 bp |
| A.F | (SEQ ID NO:3) | | human follistatin | |
| | | | gene | |
| 5kb-UP- | R:GCCTTGCTTTCCCCTTTAAT | 59.5 | | |
| A.R | (SEQ ID NO:4) | | | |
| Southern- | F:CTGGGTCACTGGTAACTGACATT | 60.3 | intron 1 of human | 506 bp |
| F | (SEQ ID NO:5) | | follistatin gene | |
| Southern- | R:GTCCTACCTTTACAGGGGATG | 60.2 | | |
| R | (SEQ ID NO:6) | | | |
| 1.5 kb | F:TTTCAAACACCATGACCCAA | 59.7 | 1.5 kb downstream | 297 bp |
| DOWN- | (SEQ ID NO:7) | | of human follistatin | |
| A.F | | | gene | |

TABLE 3

PCR primers for genotyping pups from the second
step of 15 cross-breeding

| | | | | |
|---|---|---|---|---|
| | | Tm | | Product |
| Primer | Sequence (5′ 3′) | (C) | Targeting site | size |

| | | | | |
|---|---|---|---|---|
| hHPRT.3F | F:TGCTGACCTGCTGGATTACA | 60.4 | the sequence in the | 208 bp |
| | (SEQ ID NO:8) | | replacement cassette | |
| hHPRT.3R | R:CTGCATTGTTTTGCCAGTGT | 59.7 | in follistatin knock- | |
| | (SEQ ID NO:9) | | out mice | |
| human.FS.2F | F:ACTGCTCACTCACCCACCTC | 60.3 | the human follistatin | 260 bp |
| | (SEQ ID NO:10) | | gene | |
| human.FS.2R | R:CTGCAAGTTGGGAAGAAGGA | 60.3 | | |
| | (SEQ ID NO:11) | | | |
| mouse.FS.2F | F:TGTGCCTCTTTCCAACTCCT | 59.8 | the mouse follistatin | 305 bp |
| | (SEQ ID NO:12) | | gene | |

TABLE 4

Primers of the real-time PCR assays for human and
mouse follistatin cDNA, and mouse J3 actin cDNA

| | | | | |
|---|---|---|---|---|
| | | Tm | Targeting | Product |
| Primer | Sequence (5′ 3′) | (C) | Site | Size |

| | | | | |
|---|---|---|---|---|
| Human.screen.2F | F:TGTGGTGGACCAGACCAATA | 59.8 | Human | 101 bp |
| | (SEQ ID NO:13) | | follistatin | |
| | | | cDNA | |
| Human.screen.2R | R:TGACTCCATCATTCCCACAG | 59.5 | | |
| | (SEQ ID NO:14) | | | |
| Mouse.screen.2F | F:CCAGACTGTTCCAACATCACC | 60.4 | Mouse | 112 bp |
| | (SEQ ID NO:15) | | follistatin | |
| | | | cDNA | |
| Mouse.screen.2R | R:CTAGTTCCGGCTGCTCTTTG | 60.2 | | |
| | (SEQ ID NO:16) | | | |
| b-actin F | F:GCTACAGCTTCACCACCACA | 59.9 | Mouse β | 208 bp |
| | (SEQ ID NO:17) | | actin cDNA | |
| b-actin.R | R:AAGGAAGGCTGGAAAAGAGC | 60.0 | | |
| | (SEQ ID NO:18) | | | |

TABLE 5
Conditions of the real-time PCR using the LightCyclerTM for the
primers “human.screen.2”, “mouse.screen.2” and “b-actin”

PCR primers:

| | human.screen.2 | mouse.screen.2 | b-actin |
|---|---|---|---|
| Reaction volume | 20 μl | 20 μl | 20 μl |
| PCR Master Mix | | | |
| Primers | 0.5 μM | 0.5 μM | 0.5 μM |
| MgCI2 | 4 mM | 4 mM | 4 mM |
| SYBR green I | 2 μI | 2 μI | 2 μI |
| PCR protocol: | | | |
| heating/cooling rate | 20 C/sec | 20 C/sec | 20 C/sec |
| 1. pre-incubation: | 95 C/10 min | 95 C/10 min | 95 C/10 min |
| number of cycles | 1 | 1 | 1 |
| 2. amplification: | | | |
| denaturation | 95 C/10 sec | 95 C/10 sec | 95 C/10 sec |
| anealing | 59 C/3 sec | 63 C/2 sec | 58 C/3 sec |
| extension | 72 C/5 sec | 72 C/5 sec | 72 C/9 sec |
| acquisition mode | single | single | single |
| number of cycles | 45 | 45 | 45 |
| 3. melting: | | | |
| melting temperature | 65 C/15 sec | 65 C/15 sec | 65 C/15 sec |
| acquisition mode | continuous | continuous | continuous |
| melting | 95 C with | 95 C with | 95 C with |
| | 0.1 C/sec | 0.1 C/sec | 0.1 C/sec |
| 4. cooling | 40 C/30 sec | 40 C/30 sec | 40 C/30 sec |

TABLE 6
Pronuclear microinjection data for pNEB-FS and PAC-FS 15 vectors

| | No. of | No. of | No. of | |
|---|---|---|---|---|
| | zygotes with | 1-cell embryos | fosters | Pups |
| | two pronuclei | transferred | used | born |
| pNEB-FS | 323 | 208 | 13 | 36 |
| PAC-FS | 1353 | 810 | 37 | 89 |

TABLE 7
The ratios of mRNA expression levels of human follistatin transgenes to
mouse endogenous gene (human follistatin mRNA I mouse follistatin mRNA) in
different tissues of independent transgenic lines

| | ovary | testis | muscle | kidney | liver | brain |
|---|---|---|---|---|---|---|
| FS⁹⁵ -wt.1 | 8.9E−05 | 3.8E−02 | 1.8E−00 | 6.2E−03 | 1.4E−02 | 8.3E−02 |
| FS⁹⁵ -wt.2 | 5.5E−04 | 1.7E−02 | 1.3E−01 | 2.2E−01 | 1.7E−02 | 4.0E−02 |
| FS -wt.1 | 6.9E−04 | 3.4E−02 | 8.5E−02 | 2.7E−02 | 4.8E−02 | 2.1E−02 |
| FS -wt.2 | 9.3E−05 | 4.4E−02 | 5.2E−01 | 6.5E−03 | 8.0E−02 | 1.1E−02 |

TABLE 8
Pronuclear microinjection data for the transgene constructs PAC-FSm1
and PAC-FSm2

| | No. of zygotes | No. of 1-cell | No. of | No. of | No. of |
|---|---|---|---|---|---|
| | of pronuclear | embryos | fosters | pups | foun- |
| Construct | microinjection | transferred | used | born | ders |
| PAC-FSm1 | 359 | 211 | 11 | 8 | 3 |
| PAC-FSm2 | 810 | 545 | 23 | 48 | 1 |

TABLE 9
Weights and crown-rump lengths (CRL) at birth of rescued mouse lines
and their wild-type and knockout littermates

| | | Weights (gm) | CRL (cm) |
|---|---|---|---|
| | | (mean ± standard | (mean ± standard |
| Mouse line | Number | deviation) | deviation) |
| FS⁹⁵-288-ko.1 | 9 | 1.11 ± 0.11^{d} | 2.12 :±: 0.13^{c,e} |
| FS⁹⁵-288-ko.2 | 9 | 1.12 ± 0.09^{c} | 2.11 ± 0.17^{d,f} |
| FS⁹⁵-315-ko.1 | 7 | 1.19 ± 0.07^{a} | 2.49 ± 0.07^{a,e,f} |
| fs-ko | 10 | 1.01 ± 0.10^{a,b} | 2.02 ±: 0.08^{a,b} |
| wt | 10 | 1.29 ± 0.09^{b,c.d} | 2.63 ± 0.08^{b,c,d} |

(a, b, c, d, e and f represent statistical significance of p < 0.05.)

### BIBLIOGRAPHY

Bunin B A, et al. (1994) Proc. Natl. Acad. Sci. USA, 91:4708-4712
Camper and Saunders, 2000
Cancilla B, Jarred R A, Wang H, Mellor S L, Cunha G R and Risbridger G P, (2001), Regulation of prostate branching morphogenesis by activin A and follistatin, Developmental Biology 237 145-158
Chang H, Brown C W and Matzuk M M (2002) Genetic analysis of the mammalian transforming growth factor-beta superfamily. Endocr Rev 23 787-823
DeWitt S H, et al. (1993) Proc. Natl. Acad. Sci. USA, 90:6909-6913
Iemura S, Yamamoto T S, Takagi C, Uchiyama H, Natsume T, Shimasaki S, Sugino H and Ueno N (1998), Direct binding of follistatin to a complex of bone-morphogenetic protein and its receptor inhibits ventral and epidermal cell fates in early Xenopus embryo, Proceedings of the National Academy of Sciences of the United States of America 95 9337-9342
Lee S J and McPherran A C (2001), Regulation of myostatin activity and muscle growth Proceedings of the National Academy of Sciences of the United States of America 98 9306-9311
Lin S Y, Morrison J R, Phillips D J and De Kretser D M (2003) Regulation of ovarian function by the TGF-beta superfamily and follistatin. Reproduction 126 133-148
Maeshima A, Nojima Y and Kojima I, (2001) The role of the activin-follistatin system in the development and regeneration processes of the kidney, Cytokine Growth Factor Rev 12 289-298
Matzuk M M, Lu N, Vogel H, Sellheyer K, Roop D R and Bradley A (1995) Multiple defects and perinatal death in mice deficient in follistatin, Nature 374 360-363
Otsuka F, Moore R K, Iemura Si S, Ueno N and Shimasaki S (2001) Follistatin Inhibits the Function of the Oocyte-Derived Factor BMP-15. Biochem Biophys Res Commun 289 961-966
Shimasaki, et al., 1988, Primary structure of the human follistatin precursor and its genomic organization Proceedings of the National Academy of Sciences of the United States of America 85 4218-4222
Stein and Cohen, 1988, Cancer Res 48:2659-68
van der Krol et al., 1988, Biotechniques 6:958-976
Welt C, Sidis Y, Keutmann H and Schneyer A (2002) Activins, inhibins, and follistatins: from endocrinology to signaling. A paradigm for the new millennium. Exp Biol Med (Maywood) 227 724-752
Yang, N., (1992) Gene Transfer into Mammalian Somatic Cells in vivo, Grit. Rev. Biotechn. 12(4): 335-356

1-41. (canceled)

42. A method of modulating growth of tissue in a mammal, comprising administering to the mammal an agent in an amount sufficient to provide the effects of a functionally effective level of follistatin 315 in the mammal.

43. The method of claim 42, wherein the agent is natural, recombinant or synthetic follistatin 315.

44. The method of claim 42, wherein the agent is a modulatory agent with an in vivo activity of a functionally effective level of follistatin 315.

45. The method of claim 44, wherein the modulatory agent is a monoclonal antibody which is an antagonist of activin A.

46. A method of modulating the biological functions of cells in a mammal, comprising the step of administering to the mammal an activin A antagonist, wherein the antagonist is capable of eliciting the desired biological function of a functionally effective level of follistatin 315.

47. The method of claim 46, wherein the activin A antagonist is natural, recombinant or synthetic follistatin 315.

48. The method of claim 46, wherein the activin A antagonist is a small molecule.

49. The method of claim 46, wherein the activin A antagonist is a peptide mimetic.

50. The method of claim 47, wherein the activin A antagonist is delivered to the mammal as a nucleic acid therapeutic.

51. A modulatory agent, wherein the modulatory agent has an in vivo activity of a functionally effective level of follistatin 315.

52. The agent of claim 51, wherein the agent is non-proteinaceous.

53. The agent of claim 51, wherein the agent comprises proteinaceous elements.

54. The agent of claim 51, wherein the agent has a modified side chain.

55. The agent of claim 51, wherein the agent comprises crosslinkers which impose conformational constraints on the agent.

56. The agent of claim 52, wherein the agent is a monoclonal antibody which is an antagonist of activin A.

57. The agent of claim 52, wherein the agent is comprised of active regions of follistatin 315.

58. The agent of claim 52, wherein the agent comprises the entire follistatin 315 peptide fused to another molecule.

59. The agent of claim 52, wherein the agent comprises modified amino acids.

60. A pharmaceutical composition comprising the modulatory agent of claim 51 and an excipient.

61. A cell characterised by increased levels of follistatin 315 as a result of delivery of follistatin 315 to the cell.

62. The cell of claim 61 wherein said cell is transfected with a gene encoding follistatin 315.

63. The cell of claim 62 wherein the gene encoding follistatin 315 is constitutively expressed.
